SEQUENCE LISTING (47 sequences)

SEQ ID NO: 1 - protein, 152 aa, Escherichia coli [sequence omitted]

SEQ ID NO: 2 - protein, 326 aa, Escherichia coli
MKKIPLGTTDITLSRMGLGTWAIGGGPAWNGDLDRQICIDTILEAHRCGINLIDTAPGYNFGNSEVIVGQALKKLPREQVVVETKCGIVWERKGSLFNKVGDRQLYKNLSPESIREEVAASLQRLGIDYIDIYMTHWQSVPPFFTPIAETVAVLNELKSEGKIRAIGAANVDADHIREYLQYGELDIIQAKYSILDRAMENELLPLCRDNGIVVQVYSPLEQGLLTGTITRDYVPGGARANKVWFQRENMLKVIDMLEQWQPLCARYQCTIPTLALAWILKQSDLISILSGATAPEQVRENVAALNINLSDADATLMREMAEALER

SEQ ID NO: 3 - protein, 367 aa, Escherichia coli
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 4 - protein, 256 aa, Klebsiella pneumoniae
MKKVALVTGAGQGIGKAIALRLVKDGFAVAIADYNDTTAKAVASEINQAGGRAMAVKVDVSDRDQVFAAVEQARKTLGGFDVIVNNAGVAPSTPIESITPEIVDKVYNINVKGVIWGIQAAVEAFKKEGHGGKIINACSQAGHVGNPELAVYSSSKFAVRGLTQTAARDLAPLGITVNGYCPGIVKTPMWAEIDRQVSEAAGKPLGYGTAEFAKRITLGRLSEPEDVAACVSYLASPDSDYMTGQSLLIDGGMVFN

SEQ ID NO: 5 - protein, 554 aa, Klebsiella oxytoca
MRSKRFEALAKRPVNQDGFVKEWIEEGFIAMESPNDPKPSIKIVNGAVTELDGKPVSDFDLIDHFIARYGINLNRAEEVMAMDSVKLANMLCDPNVKRSEIVPLTTAMTPAKIVEVVSHMNVVEMMMAMQKMRARRTPSQQAHVTNVKDNPVQIAADAAEGAWRGFDEQETTVAVARYAPFNAIALLVGSQVGRPGVLTQCSLEEATELKLGMLGHTCYAETISVYGTEPVFTDGDDTPWSKGFLASSYASRGLKMRFTSGSGSEVQMGYAEGKSMLYLEARCIYITKAAGVQGLQNGSVSCIGVPSAVPSGIRAVLAENLICSSLDLECASSNDQTFTHSDMRRTARLLMQFLPGTDFISSGYSAVPNYDNMFAGSNEDAEDFDDYNVIQRDLKVDGGLRPVREEDVIAIRNKAARALQAVFAGMGLPPITDEEVEAATYAHGSKDMPERNIVEDIKFAQEIINKNRNGLEVVKALAQGGFTDVAQDMLNIQKAKLTGDYLHTSAIIVGDGQVLSAVNDVNDYAGPATGYRLQGERWEEIKNIPGALDPNEID

SEQ ID NO: 6 - protein, 224 aa, Klebsiella oxytoca
MEINEKLLRQIIEDVLSEMKGSDKPVSFNAPAASAAPQATPPAGDGFLTEVGEARQGTQQDEVIIAVGPAFGLAQTVNIVGIPHKSILREVIAGIEEEGIKARVIRCFKSSDVAFVAVEGNRLSGSGISIGIQSKGTTVIHQQGLPPLSNLELFPQAPLLTLETYRQIGKNAARYAKRESPQPVPTLNDQMARPKYQAKSAILHIKETKYVVTGKNPQELRVAL

SEQ ID NO: 7 - protein, 173 aa, Klebsiella oxytoca
MNTDAIESMVRDVLSRMNSLQGEAPAAAPAAGGASRSARVSDYPLANKHPEWVKTATNKTLDDFTLENVLSNKVTAQDMRITPETLRLQASIAKDAGRDRLAMNFERAAELTAVPDDRILEIYNALRPYRSTKEELLAIADDLESRYQAKICAAFVREAATLYVERKKLKGDD

SEQ ID NO: 8 - protein, 555 aa, Klebsiella pneumoniae
MKRSKRFAVLAQRPVNQDGLIGEWPEEGLIAMDSPFDPVSSVKVDNGLIVELDGKRRDQFDMIDRFIADYAINVERTEQAMRLEAVEIARMLVDIHVSREEIIAITTAITPAKAVEVMAQMNVVEMMMALQKMRARRTPSNQCHVTNLKDNPVQIAADAAEAGIRGFSEQETTVGIARYAPFNALALLVGSQCGRPGVLTQCSVEEATELELGMRGLTSYAETVSVYGTEAVFTDGDDTPWSKAFLASAYASRGLKMRYTSGTGSEALMGYSESKSMLYLESRCIFITKGAGVQGLQNGAVSCIGMTGAVPSGIRAVLAENLIASMLDLEVASANDQTFSHSDIRRTARTLMQMLPGTDFIFSGYSAVPNYDNMFAGSNFDAEDFDDYNILQRDLMVDGGLRPVTEAETIAIRQKAARAIQAVFRELGLPPIADEEVEAATYAHGSNEMPPRNVVEDLSAVEEMMKRNITGLDIVGALSRSGFEDIASNILNMLRQRVTGDYLQTSAILDRQFEVVSAVNDINDYQGPGTGYRISAERWAEIKNIPGVVQPDTIE

SEQ ID NO: 9 - protein, 146 aa, Klebsiella pneumoniae
MPHGAILKELIAGVEEEGLHARVVRILRTSDVSFMAWDAANLSGSGIGIGIQSKGTTVIHQRDLLPLSNLELFSQAPLLTLETYRQIGKNAARYARKESPSPVPVVNDQMVRPKFMAKAALFHIKETKHVVQDAEPVTLHVDLVRE

SEQ ID NO: 10 - protein, 141 aa, Klebsiella pneumoniae
MSEKTMRVQDYPLATRCPEHILTPTGKPLTDITLEKVLSGEVGPQDVRISRQTLEYQAQIAEQMQRHAVARNFRRAAELIAIPDERILAIYNALRPFRSSQAELLAIADELEHTWHATVNAAFVRESAEVYQQRHKLRKGS

SEQ ID NO: 11 - protein, 787 aa, Clostridium butyricum
MISKGFSTQTERINILKAQILNAKPCVESERAILITESFKQTEGQPAILRRALALKHILENIPITIRDQELIVGSLTKEPRSSQVFPEFSNKWLQDELDRLNKRTGDAFQISEESKEKLKDVFEYWNGKTTSELATSYMTEETREAVNCDVFTVGNYYYNGVGHVSVDYGKVLRVGFNGIINEAKEQLEKNRSIDPDFIKKEKFLNSVIISCEAAITYVNRYAKKAKEIADNTSDAKRKAELNEIAKICSKVSGEGAKSFYEACQLFWFIHAIINIESNGHSISPARFDQYMYPYYENDKNITDKFAQELIDCIWIKLNDINKVRDEISTKHFGGYPMYQNLIVGGQNSEGKDATNKVSYMALEAAVHVKLPQPSLSVRIWNKTPDEFLLRAAELTREGLGLPAYYNDEVIIPALVSRGLTLEDARDYGIIGCVEPQKPGKTEGWHDSAFFNLARIVELTINSGFDKNKQIGPKTQNFEEMKSFDEFMKAYKAQMEYFVKHMCCADNCIDIAHAERAPLPFLSSMVDNCIGKGKSLQDGGAEYNFSGPQGVGVANIGDSLVAVKKIVFDENKITPSELKKTLNNDFKNSEEIQALLKNAPKFGNDIDEVDNLAREGALVYCREVNKYTNPRGGNFQPGLYPSSINVYFGSLTGATPDGRKSGQPLADGVSPSRGCDVSGPTAACNSVSKLDHFIASNGTLFNQKFHPSALKGDNGLMNLSSLIRSYFDQKGFHVQFNVIDKKILLAAQKNPEKYQDLIVRVAGYSAQFISLDKSIQNDIIARTEHVM

SEQ ID NO: 12 - protein, 304 aa, Clostridium butyricum
MSKEIKGVLFNIQKFSLHDGPGIRTIVFFKGCSMSCLWCSNPESQDIKPQVMFNKNLCTKCGRCKSQCKSAAIDMNSEYRIDKSKCTECTKCVDNCLSGALVIEGRNYSVEDVIKELKKDSVQYRRSNGGITLSGGEVLLQPDFAVELLKECKSYGWHTAIETAMYVNSESVKKVIPYIDLAMIDIKSMNDEIHRKFTGVSNEIILQNIKLSDELAKEIIIRIPVIEGFNADLQSIGAIAQFSKSLTNLKRIDLLPYHNYGENKYQAIGREYSLKELKSPSKDKMERLKALVEIMGIPCTIGAE

SEQ ID NO: 13 - DNA, 47 bp, artificial
  synthetic oligonucleotide primer
gggaaaggtaccatggcacttataatgaatagtaaaaaaaagatagc

SEQ ID NO: 14 - DNA, 47 bp, artificial
  synthetic oligonucleotide primer
gggaaagcatgcttaaaaattgtcttttctaattttttggtaataat

SEQ ID NO: 15 - DNA, 37 bp, artificial
  synthetic oligonucleotide primer
gggaaaggtaccatgaaaattgctttgatcgcgcatg

SEQ ID NO: 16 - DNA, 37 bp, artificial
  synthetic oligonucleotide primer
gggaaagcatgcttatacattcggctcttctccccga

SEQ ID NO: 17 - DNA, 47 bp, artificial
  synthetic oligonucleotide primer
gggaaaggtaccatgaatatagcattagtagcacatgaccaaatgaa

SEQ ID NO: 18 - DNA, 47 bp, artificial
  synthetic oligonucleotide primer
gggaaagcatgcttaaatacgttgacttttgctttttctaacttctc

SEQ ID NO: 19 - DNA, 32 bp, artificial
  synthetic oligonucleotide primer
gggaaaggtaccatggaactgacgactcgcac

SEQ ID NO: 20 - DNA, 32 bp, artificial
  synthetic oligonucleotide primer
gggaaagcatgcttacttcagacggtccgcga

SEQ ID NO: 21 - DNA, 32 bp, artificial
  synthetic oligonucleotide primer
gggaaaggtaccatgcccatgaaggccctggc

SEQ ID NO: 22 - DNA, 32 bp, artificial
  synthetic oligonucleotide primer
gggaaagcatgcctattggggggttcccttgc

SEQ ID NO: 23 - DNA, 47 bp, artificial
  synthetic oligonucleotide primer
gggaaaggtaccatgtggaatgaaaatatggaactgacaacacgtac

SEQ ID NO: 24 - DNA, 32 bp, artificial
  synthetic oligonucleotide primer
gggaaagcatgcttatttcaggcgctcggcaa

SEQ ID NO: 25 - DNA, 34 bp, artificial
  synthetic oligonucleotide primer
gggaaatgtacaatgatcggtatcagtttcaccc

SEQ ID NO: 26 - DNA, 33 bp, artificial
  synthetic oligonucleotide primer
gggaaagcatgcttatcctcggccggccaggta

SEQ ID NO: 27 - DNA, 38 bp, artificial
  synthetic oligonucleotide primer
gggaaaggtaccatgactcgcccccgcatcgcgttgat

SEQ ID NO: 28 - DNA, 32 bp, artificial
  synthetic oligonucleotide primer
gggaaatctagatcagctggccgccgcttcgt

SEQ ID NO: 29 - DNA, 49 bp, artificial
  synthetic oligonucleotide primer
gggaaagcatgcaggagatataccatggaccgcattattcaatcaccgg

SEQ ID NO: 30 - DNA, 37 bp, artificial
  synthetic oligonucleotide primer
gggaaatctagattattcccactcttgcaggaaacgc

SEQ ID NO: 31 - DNA, 40 bp, artificial
  synthetic oligonucleotide primer
gggaaaggtaccatgaaaaagatacctttaggcacaacgg

SEQ ID NO: 32 - DNA, 40 bp, artificial
  synthetic oligonucleotide primer
gggaaatctagattaacgctccagggcctctgccatttcc

SEQ ID NO: 33 - DNA, 40 bp, artificial; identical to SEQ ID NO: 31
  synthetic oligonucleotide primer

SEQ ID NO: 34 - DNA, 37 bp, artificial
  synthetic oligonucleotide primer
gggaaagtcgacttaacgctccagggcctctgccatt

SEQ ID NO: 35 - DNA, 50 bp, artificial
  synthetic oligonucleotide primer
gggaaagtcgacaggagatataccatgaaaaaagtcgcacttgttaccgg

SEQ ID NO: 36 - DNA, 37 bp, artificial
  synthetic oligonucleotide primer
gggaaactgcagttagttaaacaccatcccgccgtcg

SEQ ID NO: 37 - DNA, 51 bp, artificial
  synthetic oligonucleotide primer
gggaaactgcagaggagatataccatgaaaattgctttgatcgcgcatgac

SEQ ID NO: 38 - DNA, 37 bp, artificial
  synthetic oligonucleotide primer
gggaaatctagattatacattcggctcttctccccga

SEQ ID NO: 39 - DNA, 47 bp, artificial
  synthetic oligonucleotide primer
gggaaacgtacgatgagatcgaaaagatttgaagcactggcgaaacg

SEQ ID NO: 40 - DNA, 46 bp, artificial
  synthetic oligonucleotide primer
gggaaaaagcttttaatcgtcgcctttgagttttttacgctcgacg

SEQ ID NO: 41 - DNA, 38 bp, artificial
  synthetic oligonucleotide primer
gggaaaggatccgaaaaaagtcgcacttgttaccggcg

SEQ ID NO: 42 - DNA, 37 bp, artificial
  synthetic oligonucleotide primer
gggaaagtcgacttagttaaacaccatcccgccgtcg

SEQ ID NO: 43 - DNA, 47 bp, artificial
  synthetic oligonucleotide primer
gggcccggtaccatgaaaagatcaaaacgatttgcagtactggccca

SEQ ID NO: 44 - DNA, 47 bp, artificial
  synthetic oligonucleotide primer
gggcccaagcttttagcttcctttacgcagcttatgccgctgctgat

SEQ ID NO: 45 - DNA, 39 bp, artificial
  synthetic oligonucleotide primer
gggaaaggtaccatgatcagcaaagggttcagcacccag

SEQ ID NO: 46 - DNA, 47 bp, artificial
  synthetic oligonucleotide primer
gggaaaaagcttttattccgcgcctatagtacacggaatgcccataa

SEQ ID NO: 47 - DNA, 6 bp, artificial
  ribosomal binding site
aggaga

(19) United States
(12) Patent Application Publication
(10) Pub. No.: US 2014/0134690 A1
(43) Pub. Date: May 15, 2014

(54) MICROBES AND METHODS FOR PRODUCING 1-PROPANOL

(71) Applicant: University of Georgia Research Foundation, Inc., Athens, GA (US)

(72) Inventors: YAJUN YAN, ATHENS, GA (US); RACHIT JAIN, ATHENS, GA (US)

(73) Assignee: University of Georgia Research Foundation, Inc., Athens, GA (US)

(21) Appl. No.: 14/072,102

(22) Filed: Nov. 5, 2013

Related U.S. Application Data
(60) Provisional application No. 61/723,007, filed on Nov. 6, 2012.

(51) Int. Cl. C12P 7/04

(52) U.S. Cl. CPC C12P 7/04
USPC 435/157; 435/252.33

(57) ABSTRACT

Provided herein are microbes metabolically engineered to produce 1-propanol from a 1,2-propanediol intermediate. The microbes may include one or two pathways for production of 1-propanol from a 1,2-propanediol intermediate. Also provided herein are methods for using the microbes for the production of 1-propanol.

### CROSS-REFERENCE TO RELATED APPLICATIONS

This application claims the benefit of U.S. Provisional Application Ser. No. 61/723,007, filed Nov. 6, 2012, which is incorporated by reference herein.

### GOVERNMENT FUNDING

The present invention was made with government support under 1335856, awarded by the National Science Foundation. The government has certain rights in the invention.

### BACKGROUND

The excessive utilization of petroleum plays a major role in the release of the greenhouse gas-carbon dioxide contributing to global warming. Renewable energy sources provide a wide platform of resources to address the problem of increasing energy demand The manufacture of biofuels such as higher chain alcohols from renewable sources provides an alternative energy source which possesses the advantage of having desirable fuel properties and uncomplicated transportability (Atsumi et al., Nature 2008, 451:86-89; Atsumi and Liao, Appl. Environ. Microbiol. 2008, 74:7802-7808; Atsumi and Liao, Curr. Opin. Biotech. 2008, 19:414-419; Connor and Atsumi, J. Biomed. Biotech. 2010:541698). The synthesis of various higher chain alcohols has been achieved by constructing biosynthetic pathways in E. coli and other micro-organisms (Atsumi et al., Nature 2008, 451:86-89; Atsumi and Liao, Appl. Environ. Microbiol. 2008, 74:7802-7808; Atsumi and Liao, Curr. Opin. Biotechn. 2008, 19:414-419; Connor and Atsumi, J. Biomed. Biotech. 2010:541698; Atsumi et al., Metab. Eng. 2008, 10:305-311; Inokuma et al., J. Biosci. Bioeng. 2010, 110:696-701; Shen and Liao, Metab. Eng. 2008, 10:312-320). Here, we describe the design of a new pathway for 1-propanol synthesis and its validation in E. coli.

In the petrochemical industry, 1-propanol is produced from ethene by a reaction with carbon monoxide and hydrogen to give propionaldehyde, which is then hydrogenated (International Programme on Chemical Safety, “Environmental Health Criteria 102. 1-Propanol,” available online at www.inchem.org/documents/ehc/ehc/ehc102.htm). 1-propanol is also produced as a by-product when potatoes or grains are fermented during the commercial manufacture of ethanol (International Programme on Chemical Safety, “Environmental Health Criteria 102. 1-Propanol,” available online at www.inchem.org/documents/ehc/ehc/ehc102.htm; Material Safety Data Sheet 1-Propanol, Calcdon Laboratory chemicals, available online at www.calcdonlabs.com/upload/msds/8500-1e.pdf).

The general use of 1-propanol is in the manufacture of drugs and cosmetics such as lotions, soaps, and nail polishes. It also finds applications in the manufacture of flexographic printing ink and textiles (International Programme on Chemical Safety, “Environmental Health Criteria 102. 1-Propanol,” available online at www.inchem.org/documents/ehc/ehc/ehc102.htm; Material Safety Data Sheet 1-Propanol, Calcdon Laboratory chemicals, available online at www.calcdonlabs.com/upload/msds/8500-1e.pdf).

Recently, the use of 1-propanol as a potential fuel substitute to petroleum has promoted the interest in its production via biological approaches. In 2008, Atsumi et al. and Shen et al. reported the production of 1-propanol from glucose by metabolic engineering of E. coli. Their work relied on the keto-acid pathway in E. coli with 2-ketobutyrate as a key intermediate (Atsumi et al., Nature 2008, 451:86-89; Shen and Liao, Metab. Eng. 2008, 10:312-320). The 2-ketobutyrate was converted to 1-propanol by the action of a keto acid decarboxylase and an alcohol dehydrogenase. Wild type E. coli carrying this pathway was able to produce around 0.15 g/L of 1-propanol. With the elimination of the genes metA, tdh, ilvB, ilvl and adhE encoding the enzymes o-succinyltransferase, threonine dehydrogenase, acetohydroxy acid synthase and alcohol dehydrogenase, respectively, the production of 1-propanol achieved was 1 g/L. Atsumi et al. (Atsumi and Liao, Appl. Environ. Microbiol. 2008, 74:7802-7808) reported higher levels of 1-propanol production in E. coli using cimA encoding a citramalate synthase from Methanoccus jannaschii. They established a direct route for the conversion of pyruvate to 2-ketobutyrate. With the utilization of citramalate pathway and incorporating an evolutionary strategy based on growth they were able to overcome feedback inhibition by isoleucine. Using wild type cimA they achieved 0.3 g/L of 1-propanol production. With the development of cimA variants, the production of 1-propanol was 9 times higher compared to the wild type cimA.

The pathway that leads to the synthesis of 1,2-propanediol has been introduced by others into both E. coli and Saccharomyces cerevisiae. By over-expressing the E. coli genes mgsA (encoding methylglyoxal synthase) and gldA (encoding glycerol dehydrogenase) and relying on the native expression of other enzymes, Altaras et al. achieved the production of 0.7 g/L of 1,2-propanediol in E. coli (Appl. Environ. Microbiol. 1999, 65:1180-1185). Production of 1,2-propanediol (1.08 g/L) in E. coli was reported by Berrios-Rivera et al. by utilizing Clostridium acetobutylicum mgsA and E. coli gldA in a strain deficient in lactate production using an initial glucose concentration of 101.68 mM (J. Indust. Microbiol. Biotech. 2003, 30:34-40). Enhanced production of 1,2-propanediol in E. coli was also reported by Altaras et al. (Biotech. Prog. 2000, 16:940-946). The study involved expression of more complete pathway by addition of fucO gene (1,2-propanediol oxidoreductase) responsible for the conversion of lactaldehyde to 1,2-propanediol and deletion of the competing pathway for lactate which involves the gene ldhA. Shake flask fermentation with the ldhA-strain carrying the pathway led to the production of 1,2-propanediol at a titer of 1.27 g/L, while fed-batch fermentation gave a result of 4.5 g/L of 1,2-propanediol. 1,2-propanediol production in S. cerevisiae was achieved by Joon-Young et al. (J. Microbiol. Biotech. 2008, 18:1797-1802). Their strategy was based on the idea of channeling the carbon flux towards dihydroxyacetone phosphate with the deletion of triosphoshate isomerase in S. cerevisiae via triple homologous recombination. With the introduction of 1,2-propanediol pathways consisting of the E. coli genes mgsA and gldA, the engineered S. cerevisiae produced 1.11 g/L of 1,2-propanediol compared to 0.89 g/L produced from the strain lacking the gene tpiI.

### SUMMARY OF THE INVENTION

Producing commodity chemicals from renewable and low-value sources through microbial fermentation is an alternative to the current petroleum-based chemical industry. The invention described here concerns the construction of a metabolic pathway for production of a commercial commodity, 1-propanol, from glucose using recombinant microorganisms. The pathway for 1-propanol production has been initially designed and constructed in E. coli, and production of 1-propanol from glucose has been achieved. The product 1-propanol has broad applications in the biofuel industry and in the manufacture of drugs and cosmetics. Advantageously, this technology allows the use of large scale fermentation using engineered microbes for 1-propanol production.

Provided herein are genetically engineered microbes. In one embodiment, a microbe is metabolically engineered to include at least one metabolic pathway for the production of 1-propanol from a 1,2-propanediol intermediate, and in one embodiment includes two metabolic pathways for the production of the intermediate, 1,2-propanediol. The 1-propanol may be produced using glucose as a carbon source. In one embodiment, the microbe is metabolically engineered to overexpress an enzyme having methylglyoxal synthase activity. In one embodiment, the microbe is metabolically engineered to overexpress an enzyme having secondary alcohol dehydrogenase activity, such as a diol dehydrogenase. In one embodiment, the microbe is metabolically engineered to overexpress an enzyme having primary alcohol dehydrogenase activity, such as a methylglyoxal reductase or a lactaldehyde reductase. In one embodiment, an overexpressed enzyme is native to the microbe.

In one embodiment, the microbe includes a first vector that includes a polynucleotide encoding at least one enzyme in a 1,2-propanediol pathway, the enzyme selected from one having methylglyoxal synthase activity, one having secondary alcohol dehydrogenase activity, and one having primary alcohol dehydrogenase activity, or a combination thereof. In one embodiment, the vector encodes an enzyme having methylglyoxal synthase activity and an enzyme having secondary alcohol dehydrogenase activity, where the enzyme having secondary alcohol dehydrogenase activity is a diol dehydrogenase and/or a glycerol dehydrogenase.

In one embodiment, the microbe is metabolically engineered to overexpress an enzyme having diol dehydratase activity, such as a propanediol dehydratase and/or a glycerol dehydratase. In one embodiment, the microbe is metabolically engineered to overexpress an enzyme having 1-propanal reductase activity.

In one embodiment, the microbe is a prokaryotic cell, such as E. coli. In one embodiment, the E. coli includes an enzyme having primary alcohol dehydrogenase activity, wherein the primary alcohol dehydrogenase is a lactaldehyde reductase, and wherein the lactaldehyde reductase is native to the cell. In one embodiment, the E. coli includes an enzyme having primary alcohol dehydrogenase activity, wherein the primary alcohol dehydrogenase is a lactaldehyde reductase, and wherein the lactaldehyde reductase is heterologous to the prokaryotic cell. In one embodiment, the E. coli includes an enzyme having primary alcohol dehydrogenase activity, wherein the primary alcohol dehydrogenase is a 1-propanal reductase, and wherein the 1-propanal reductase is native to the prokaryotic cell. In one embodiment, the E. coli includes an enzyme having primary alcohol dehydrogenase activity, wherein the primary alcohol dehydrogenase is a 1-propanal reductase, and wherein the 1-propanal reductase is heterologous to the prokaryotic cell.

Also provided herein are methods for producing 1-propanol by culturing a microbe described herein under conditions suitable to produce 1-propanol. The method may optionally include isolating the 1-propanol. In one embodiment the microbe is cultured in a low phosphate medium, and in one embodiment the microbe is cultured under anaerobic conditions.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1A shows a native metabolic pathway for 1,2-propanediol and 1-propanol production via the pyruvate pathway (left-hand pathway) or a designed metabolic pathway for 1,2-propanediol and 1-propanol production via the methylglyoxal pathway (right-hand pathway). FIG. 1B sets forth differences in each pathway.

FIG. 2A shows a designed metabolic pathway for 1,2-propanediol and 1-propanol production. Key enzymes are 1: methylglyoxal synthase (mgsA); 2: methylglyoxal reductase (ydjG); 3, 4: secondary alcohol dehydrogenase (gldA/budC); 5: primary alcohol dehydrogenase (fucO); 6: diol dehydratase (ppdABC/gldABC/dhaB12); 7: primary alcohol dehydrogenase (yqhD). Individual steps in the pathway are set forth in FIGS. 2B-2E. FIG. 2B shows the conversion of dihydroxyacetone-phosphate to methylglyoxal by a methylglyoxal synthase enzyme. FIG. 2C shows the conversion of methylglyoxal to hydroxyacetone by a primary alcohol dehydrogenase, a methylglyoxal reductase enzyme. FIG. 2D shows the dual pathway conversion of methylglyoxal to 1,2-propanediol by a secondary alcohol dehydrogenase. FIG. 2E shows the conversion of 1,2 propanediol to 1-propanol by a diol dehyratase.

FIG. 3A shows schematic plasmids for use in introducing the 1-propanol pathway into microorganisms. FIG. 3B shows specific plasmids pRJ11 and pYY93.

FIG. 4 shows results of in vivo enzyme assay of diol dehydratase using 5 g/L (65.7 mM) 1,2-propanediol as the substrate.

FIG. 5 shows schematics of plasmids for use in two alternative expression strategies for 1-propanol production.

FIG. 6 shows an alternative strain engineering strategy.

FIG. 7 presents amino acid sequences of examples of enzymes useful in various embodiments described herein. SEQ ID NO:1 is an example of a methylglyoxal synthase, SEQ ID NO:2 is an example of a methylglyoxal reductase, SEQ ID NO:3 is an example of a glycerol dehydrogenase, and SEQ ID NO:4 is an example of a diol dehydrogenase. SEQ ID NOs:5-7 are examples of the subunits of a diol dehydratase, SEQ ID NOs:8-10 are examples of the subunits of a diol dehydratase, and SEQ ID NOs:11-12 are examples of the subunits of another diol dehydratase.

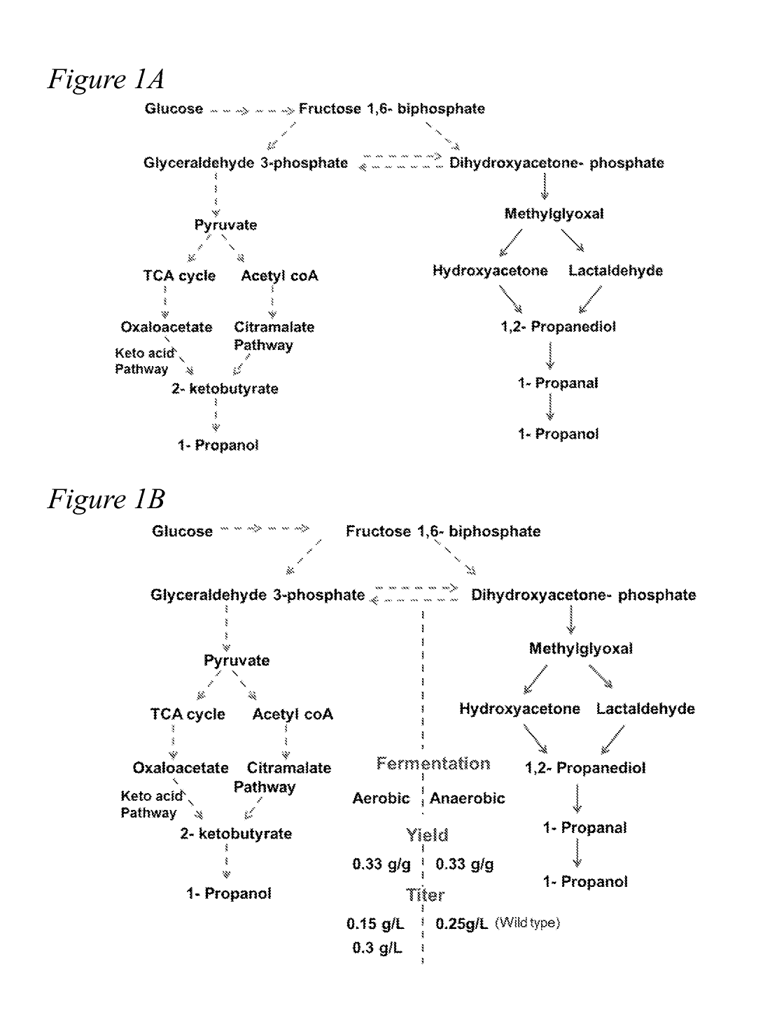

### DETAILED DESCRIPTION OF ILLUSTRATIVE EMBODIMENTS

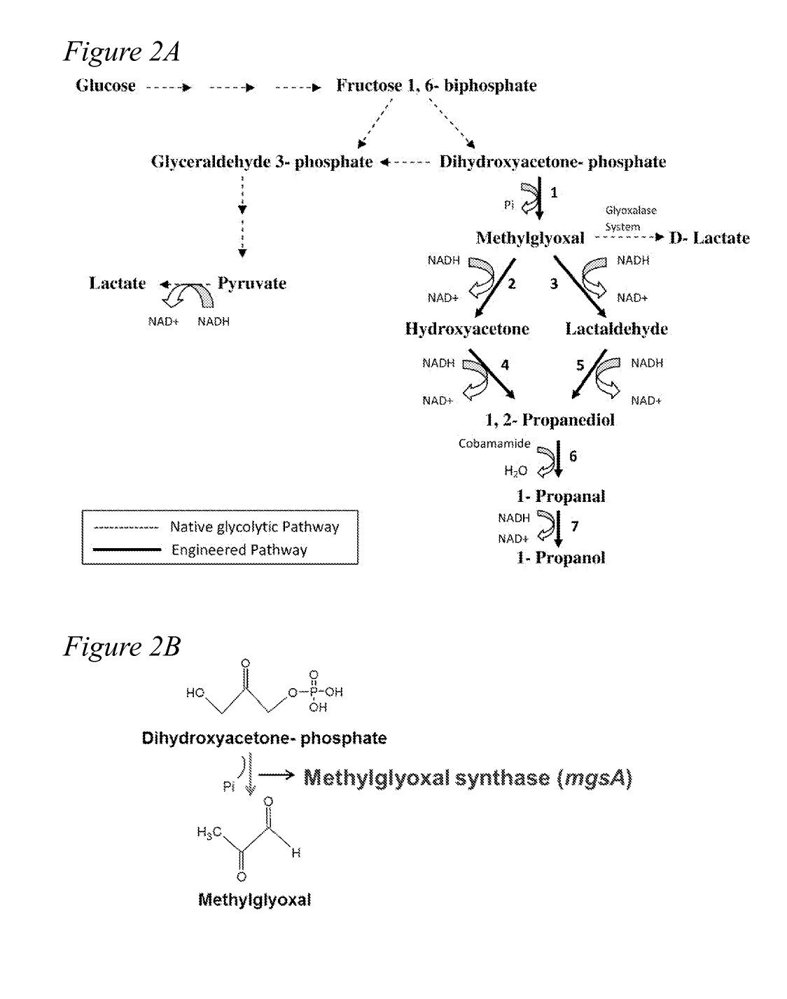

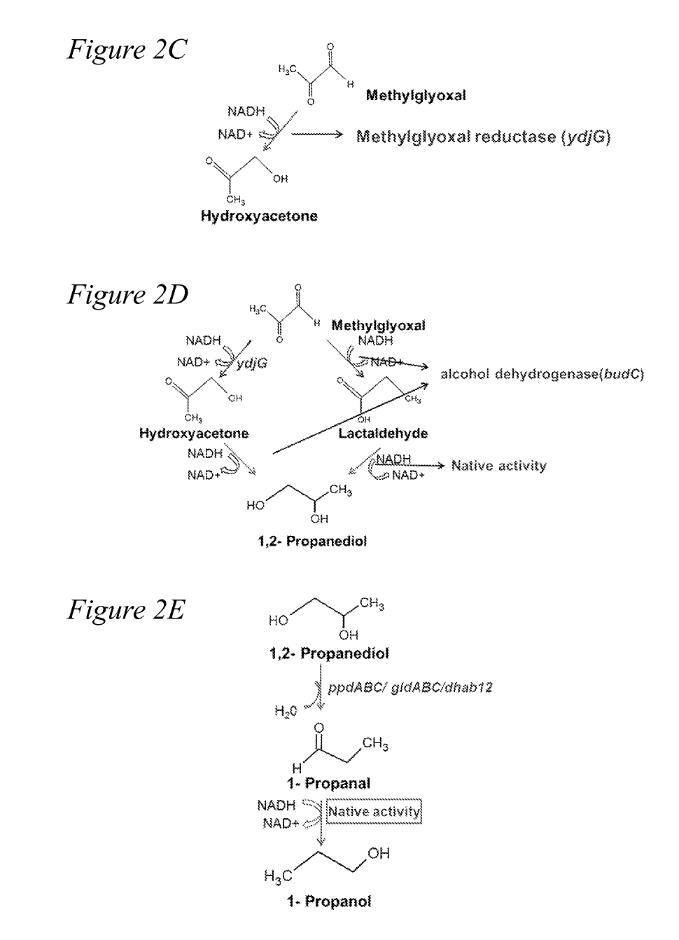

A new approach for the biosynthesis of 1-propanol has been developed by creatively exploiting and extending the existing metabolic pathway for the synthesis of 1,2-propanediol, the pathway scheme of which is shown in FIG. 2A. A glycolysis intermediate, dihydroxyacetone phosphate, can be converted to methylglyoxal by the action of the enzyme methylglyoxal synthase (FIG. 2B). The methylglyoxal generated is further reduced to either hydroxyacetone or lactaldehyde via two different routes. The formation of hydroxyacetone is catalyzed by the enzyme methylglyoxal reductase which is a primary alcohol dehydrogenase (FIG. 2C), while a secondary alcohol dehydrogenase such as glycerol dehydrogenase reduces methylglyoxal into lactaldehyde (FIG. 2D). Both hydroxyacetone and lactaldehyde can be further reduced to 1,2-propanediol. Hydroxyacetone can be reduced by a secondary alcohol dehydrogenase and lactaldehyde can be reduced by a primary alcohol dehydrogenase. The dehydration of 1,2-propanediol into 1-propanal can be achieved by a diol dehydratase (FIG. 2E). The conversion of 1-propanal to 1-propanol is also catalyzed by a primary alcohol dehydrogenase (FIG. 2E).

Provided herein are microbial cells that are metabolically engineered for production of 1-propanol; as well methods for making such cells and methods for producing and isolating 1-propanol.

In the description that follows, microbial cells are metabolically engineered to extend and exploit the native production of dihydroxyacetone-phosphate, an intermediate of glycolysis, to synthesize 1-propanol from the intermediate 1,2-propanediol by means of, in one embodiment, at least one metabolic pathway, or in a second embodiment, parallel or dual metabolic pathways. Descriptions of various embodiments refer to enzymes having different activities. These enzymes may be endogenous to the metabolically engineered cell, or may be encoded by a heterologous polynucleotide that has been introduced into the cell. In embodiments where the microbial cell natively produces an enzyme, the microbial cell may be modified to increase expression of a coding region encoding the native enzyme. In one embodiment, a promoter operably linked to the coding region may be modified using standard methods that include, for instance, homologous recombination.

As used herein, the term “polypeptide” refers broadly to a polymer of two or more amino acids joined together by peptide bonds. The term “polypeptide” also includes molecules which contain more than one polypeptide joined by disulfide bonds, ionic bonds, or hydrophobic interactions, or complexes of polypeptides that are joined together, covalently or noncovalently, as multimers (e.g., dimers, tetramers, etc.). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

As used herein, the term “polynucleotide” refers to a polymeric faun of nucleotides of any length, either ribonucleotides or deoxynucleotides, and includes both double- and single-stranded DNA and RNA. A polynucleotide may include nucleotide sequences having different functions, including for instance coding sequences, and non-coding sequences such as regulatory sequences. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment. It should be understood that sequences disclosed herein as DNA can be converted from a DNA sequence to an RNA sequence by replacing each thymidine nucleotide with a uridine nucleotide. Likewise, RNA sequences disclosed herein can be converted from an RNA sequence to a DNA sequence by replacing each uridine nucleotide with a thymidine nucleotide

As used herein, a “heterologous” polypeptide or polynucleotide refers to a polypeptide or polynucleotide that is not normally or naturally found in a microbe. An “endogenous” polypeptide or polynucleotide is also referred to as a native polypeptide or native polynucleotide.

The terms “coding region” and “coding sequence” are used interchangeably and refer to a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end

In some embodiments, a metabolically engineered cell includes at least one heterologous polynucleotide that encodes one or more polypeptides having an enzymatic activity described herein. In one embodiment, a heterologous polynucleotide may include one or more coding regions encoding one enzyme. In one embodiment, a heterologous polynucleotide may include multiple coding regions, where each coding region encodes a different enzyme. In one embodiment, the microbial cell can include a plurality of heterologous polynucleotides.

The 1,2-propanediol pathway was constructed in the wild type E. coli strain BW25113 by expressing the genes, mgsA from B. subtilis, budC from K. pneumoniae, and native E. coli ydjG. The result was the production of 1,2-propanediol at a titer of 0.8 g/L in shake flasks. We achieved the conversion of 1,2-propanediol to 1-propanol via two successive enzymatic steps by expressing the operon ppdABC from K. oxytoca and using the native activity of E. coli alcohol dehydrogenases (Jeter, J. Gen. Miicrobiol. 1990, 136:887-896; O'Brien et al., Biochem. J. 2004, 43:4635-4645; Roth et al., Ann. Rev. Microbiol. 1996, 50:137-181). This established a new pathway for 1-propanol production by manipulating the glycolytic pathway in E. coli.

The microbial pathway described herein for the production of 1-propanol from a 1,2-propanediol intermediate includes an enzyme having methylglyoxal synthase activity. As used herein, “methylglyoxal synthase” refers to a polypeptide that, regardless of its common name or native function, catalyses the conversion dihydroxyacetone-phosphate to methylglyoxal (see reaction 1 in FIG. 2A, and FIG. 2B), and a polypeptide catalysing such a conversion has methylglyoxal synthase activity. In one embodiment, such an enzyme is a member of the group having Enzyme Commission (EC) number 4.2.3.3. Enzymes having methylglyoxal synthase activity are readily available from, for instance, C. acetobutylicum (mgsA), B. subtilis (mgsA), C. difficile (mgsA), E. coli (mgsA), T. thermophilus (mgsA), K. pneumoniae (mgsA), P. fluorescens (mgsA), and R. eutropha (mgsA), and others. In one embodiment, the polypeptide having methylglyoxal synthase activity is, or is structurally similar to, a reference polypeptide that includes the amino acid sequence of SEQ ID NO:1.

As used herein, a polypeptide is “structurally similar” to a reference polypeptide if the amino acid sequence of the polypeptide possesses a specified amount of similarity and/or identity compared to the reference polypeptide. Structural similarity of two polypeptides can be determined by aligning the residues of the two polypeptides (for example, a candidate polypeptide and the polypeptide of, for example, any one of SEQ ID NO:1 through SEQ ID NO:12) to optimize the number of identical amino acids along the lengths of their sequences; gaps in either or both sequences are permitted in making the alignment in order to optimize the number of identical amino acids, although the amino acids in each sequence must nonetheless remain in their proper order.

A pair-wise comparison analysis of amino acid sequences can be carried out using the BESTFIT algorithm in the GCG package (version 10.2, Madison, Wis.). Alternatively, polypeptides may be compared using the Blastp program of the BLAST 2 search algorithm, as described by Tatiana et al., (1999 FEMS Microbiol Lett, 174:247-250), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide described herein may be selected from other members of the class to which the amino acid belongs. For example, it is well-known in the art of protein biochemistry that an amino acid belonging to a grouping of amino acids having a particular size or characteristic (such as charge, hydrophobicity, and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2. Likewise, biologically active analogs of a polypeptide containing deletions or additions of one or more contiguous or noncontiguous amino acids that do not eliminate a functional activity of the polypeptide are also contemplated.

In one embodiment, a polypeptide having an activity described herein can include a polypeptide with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to a reference amino acid sequence.

In one embodiment, a polypeptide having an activity described herein can include a polypeptide with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a reference amino acid sequence.

The microbial pathway described herein also includes enzymes having primary alcohol dehydrogenase activity.

In one embodiment, a primary alcohol dehydrogenase takes one route to the synthesis of the 1,2-propanediol intermediate by using the primary alcohol dehydrogenase methylglyoxal reductase to convert methylglyoxal to hydroxyacetone. The hydroxyacetone is then converted to 1,2-propanediol by a secondary alcohol dehydrogenase. As used herein, “methylglyoxal reductase” refers to a polypeptide that, regardless of its common name or native function, catalyses the conversion methylglyoxal to hydroxyacetone (see reaction 2 in FIG. 2A, and FIG. 2C), and a polypeptide catalysing such a conversion has methylglyoxal reductase activity. In one embodiment, such an enzyme is a member of the group having EC number 1.1.1.78. Examples of methylglyoxal reductases include, but are not limited to, ydjG (E. coli). In one embodiment, the polypeptide having methylglyoxal reductase activity is, or is structurally similar to, a reference polypeptide that includes the amino acid sequence of SEQ ID NO:2.

This route to the production of 1,2-propanediol includes a secondary alcohol dehydrogenase which converts hydroxyacetone to 1,2-propanediol. Surprisingly, enzymes catalyzing this reaction also catalyzed the conversion of methylglyoxal to lactaldehyde, a second route to the synthesis of the 1,2-propanediol intermediate. Thus, as used herein, “secondary alcohol dehydrogenase” refers to a polypeptide that, regardless of its common name or native function, catalyses the conversion hydroxyacetone to 1,2-propanediol (see reaction 4 in FIG. 2A, and FIG. 2D), and/or catalyses the conversion methylglyoxal to lactaldehyde (see reaction 3 in FIG. 2A, and FIG. 2D). A polypeptide catalysing such a conversion has secondary alcohol dehydrogenase activity. Enzymes having secondary alcohol dehydrogenase activity include, but are not limited to, glycerol dehydrogenases and diol dehydrogenases. In one embodiment, a glycerol dehydrogenase is a member of the group having EC number 1.1.1.6. In one embodiment, a diol dehydrogenase is a member of the group having EC number 1.1.1.B20. An example of a glycerol dehydrogenase is the E. coli glycerol dehydrogenase gldA. An example of a diol dehydrogenase is the K. pneumoniae diol dehydrogenase budC. In one embodiment, the polypeptide having secondary alcohol dehydrogenase activity is, or is structurally similar to, a reference polypeptide that includes the amino acid sequence of SEQ ID NO:3. In one embodiment, the polypeptide having secondary alcohol dehydrogenase activity is, or is structurally similar to, a reference polypeptide that includes the amino acid sequence of SEQ ID NO:4.

In the second route to the 1,2-propanediol intermediate, the lactaldehyde is converted to 1,2-propanediol by a primary alcohol dehydrogenase. A primary alcohol dehydrogenase that converts lactaldehyde to 1,2-propanediol is a lactaldehyde reductase. As used herein, “lactaldehyde reductase” refers to a polypeptide that, regardless of its common name or native function, catalyses the conversion lactaldehyde to 1,2-propanediol (see reaction 5 in FIG. 2A, and FIG. 2D), and a polypeptide catalysing such a conversion has lactaldehyde reductase activity. In one embodiment, such an enzyme is a member of the group having EC number 1.1.1.77. Examples of lactaldehyde reductase include, but are not limited to, E. coli (fucO) (Altras et al., Biotechnol. Progress, 2000, 16:940-946).

The microbial pathway described herein for the production of 1-propanol from a 1,2-propanediol intermediate includes an enzyme having diol dehydratase activity. As used herein, “diol dehydratase” refers to a polypeptide that, regardless of its common name or native function, catalyses the conversion of 1,2-propanediol to 1-propanal (see FIG. 2E), and a polypeptide catalysing such a conversion has diol dehydratase activity. In one embodiment, such an enzyme is a member of the group having EC number 4.2.1.28. In one embodiment, a polypeptide having diol dehydratase activity may include two or more subunits encoded by separate coding regions. Examples of diol dehydratase include, but are not limited to, a propanediol dehydratase (PPD) originating in K. oxytoca (Masuda et al., Acta Crystallogr D Biol Crystallogr, 1999, 55:907-909, Tobimatsu et al., Arch Biochem Biophys, 1997, 347:132-140), a glycerol dehydratase (GLD) from K. pneumoniae (Tobimatsu et al., Biosci Biotechnol Biochem, 1998, 62:1774-1777, Toraya et al., J Bacteriol, 1978, 135:726-729), and a glycerol dehydratase (GLD) from C. butyricum (O'Brien et al., Biochemistry 2004, 43:4635-4645, Raynaud et al., Proc Natl Acad Sci USA, 2003, 100:5010-5015, Harms et al., J Bacteriol, 1988, 170:4798-4807). In one embodiment, the subunits of a polypeptide having diol dehydratase activity is, or is structurally similar to, a reference polypeptides that include the amino acid sequence of SEQ ID NO:5, the amino acid sequence of SEQ ID NO:6, and the amino acid sequence of SEQ ID NO:7 (a PPDA subunit, a PPDB subunit, and a PPDC subunit, respectively, of a propanediol dehydratase from K. oxytoca). In one embodiment, the subunits of a polypeptide having diol dehydratase activity is, or is structurally similar to, a reference polypeptides that include the amino acid sequence of SEQ ID NO:8, the amino acid sequence of SEQ ID NO:9, and the amino acid sequence of SEQ ID NO:10 (a GLDA subunit, a GLDB subunit, and a GLDC subunit, respectively, of a glycerol dehydratase originating from K. pneumoniae). In one embodiment, the subunits of a polypeptide having diol dehydratase activity is, or is structurally similar to, a reference polypeptides that include the amino acid sequence of SEQ ID NO:11, and the amino acid sequence of SEQ ID NO:12, (subunits encoded by the dhaB1 and dhaB2 genes, respectively, of a glycerol dehydratase from C. butyricum).

The reduction of 1-propanal to 1-propanol is catalysed by a primary alcohol dehydrogenase. The primary alcohol dehydrogenase is a 1-propanal reductase. As used herein, “1-propanal reductase” refers to a polypeptide that, regardless of its common name or native function, catalyses the conversion 1-propanal to 1-propanol (see reaction 7 in FIG. 2A, and FIG. 2E), and a polypeptide catalysing such a conversion has 1-propanal reductase activity. In one embodiment, such an enzyme is a member of the group having EC number 1.1.1.2. Examples of methylglyoxal reductases include, but are not limited to, yqhD (E. coli).

The novel metabolic pathway described herein is introduced into a microbial cell using genetic engineering techniques. The term “microbe” is used interchangeably with the term “microorganism” and means any microscopic organism existing as a single cell, cell clusters, or multicellular relatively complex organisms. While certain embodiments are described using E. coli, the microbes and methods of use are not limited to E. coli and there are a number of other options for microbes suitable for engineering to produce 1-propanol and for use in the methods described herein. The suitable microbial hosts for the production of 1-propanol include, but are not limited to, a wide variety of bacteria, archaea, and yeast including members of the genera Escherichia (such as E. coli), Salmonella, Clostridium, Zymomonas, Pseudomonas (such as P. putida), Bacillus (such as B. subtilis and B. licheniformis), Rhodococcus (such as R. erythropolis), Alcaligenes (such as A. eutrophus), Klebsiella, Paenibacillus (such as P. macerans), Lactobacillus (such as L. plantarum), Enterococcus (such as E. gallinarium, E. faecalis, and E. faecium), Arthrobacter, Brevibacterium, Corynebacterium Candida, Hansenula, Pichia and Saccharomyces (such as S. cerevisiae). Host cells can be individually engineered to express one or more of the pathway enzymes as needed to complete the 1-propanol pathway as described herein; for example, they can be engineered to produce the starting material dihydroxyacetone-phosphate at greater levels. In some preferred embodiments, the host cell is a bacterial cell, such as an E. coli cell. If necessary, a coding region encoding an enzyme described herein can be modified using routine methods to reflect the codon usage bias of a microbial host cell to optimize expression of a polypeptide.

A cell that has been genetically engineered to express one or more enzyme(s) described herein for 1-propanol biosynthesis may be referred to as a “host” cell, a “recombinant” cell, a “metabolically engineered” cell, a “genetically engineered” cell or simply an “engineered” cell. These and similar terms are used interchangeably. A genetically engineered cell contains one or more heterologous polynucleotides which have been created through standard molecular cloning techniques to bring together genetic material that is not natively found together. For example, a microbe is a genetically engineered microbe by virtue of introduction of a heterologous polynucleotide. “Engineered” also includes a microbe that has been genetically manipulated such that one or more endogenous nucleotides have been altered. For example, a microbe is an engineered microbe by virtue of introduction of an alteration of endogenous nucleotides into a suitable microbe. For instance, a regulatory region, such as a promoter, could be altered to result in increased or decreased expression of an operably linked endogenous coding region. DNA sequences used in the construction of recombinant DNA molecules can originate from any species. For example, bacterial DNA may be joined with fungal DNA. Alternatively, DNA sequences that do not occur anywhere in nature may be created by the chemical synthesis of DNA, and incorporated into recombinant molecules. Proteins that result from the expression of recombinant DNA are often termed recombinant proteins. Examples of recombination are described in more detail below and may include inserting foreign polynucleotides into a cell, inserting synthetic polynucleotides into a cell, or relocating or rearranging polynucleotides within a cell. Any form of recombination may be considered to be genetic engineering and therefore any recombinant cell may also be considered to be a genetically engineered cell.

Genetically engineered cells are also referred to as “metabolically engineered” cells when the genetic engineering modifies or alters one or more particular metabolic pathways so as to cause a change in metabolism. The goal of metabolic engineering is to improve the rate and conversion of a substrate into a desired product. General laboratory methods for introducing and expressing or overexpressing native and normative proteins such as enzymes in many different cell types (including bacteria, archaea, and yeasts,) are routine and known in the art; see, e.g., Sambrook et al, Molecular Cloning: A Laboratory Manual., Cold Spring Harbor Laboratory Press (1989), and Methods for General and Molecular Bacteriology, (eds. Gerhardt et al.) American Society for Microbiology, chapters 13-14 and 16-18 (1994).

The introduction of the novel biosynthetic pathway for the production of 1-propanol into a cell involves expression or overexpression of one or more enzymes included in the novel pathway. An enzyme is “overexpressed” in a recombinant cell when the enzyme is expressed at a level higher than the level at which it is expressed in a comparable wild-type cell. In cells that do not express a particular endogenous enzyme, or in cells in which the enzyme is not endogenous (i.e., the enzyme is not native to the cell), any level of expression of that enzyme in the cell is deemed an “overexpression” of that enzyme for purposes of the present invention.

As will be appreciated by a person of skill in the art, overexpression of an enzyme can be achieved through a number of molecular biology techniques. For example, overexpression can be achieved by introducing into the host cell one or more copies of a polynucleotide encoding the desired enzyme. The polynucleotide encoding the desired enzyme may be endogenous or heterologous to the host cell. Typically, the polynucleotide is introduced into the cell using a vector. The polynucleotide may be circular or linear, single-stranded or double stranded, and can be DNA, RNA, or any modification or combination thereof. The vector can be any molecule that may be used as a vehicle to transfer genetic material into a cell. Examples of vectors include plasmids, viral vectors, cosmids, and artificial chromosomes, without limitation. Examples of molecular biology techniques used to transfer nucleotide sequences into a microorganism include, without limitation, transfection, electroporation, transduction, and transformation. These methods are routine and known in the art. Insertion of a vector into a target cell is usually called transformation for bacterial cells and transfection for eukaryotic cells, however insertion of a viral vector is often called transduction. The terms transformation, transfection, and transduction, for the purpose of the present invention, are used interchangeably herein. A polynucleotide which has been transferred into a cell via the use of a vector is often referred to as a transgene.

In one embodiment, the vector is an expression vector. An “expression vector” or “expression construct” is any vector that is used to introduce a specific polynucleotide into a target cell such that once the expression vector is inside the cell, the protein that is encoded by the polynucleotide is produced by the cellular transcription and translation machinery. Typically an expression vector includes regulatory sequences operably linked to the polynucleotide encoding the desired enzyme. Regulatory sequences are common to the person of the skill in the art and may include for example, an origin of replication, a promoter sequence, and/or an enhancer sequence. The polynucleotide encoding the desired enzyme can exist extrachromosomally or can be integrated into the host cell chromosomal DNA. Extrachromosomal DNA may be contained in cytoplasmic organelles, such as mitochondria in eukaryotes. More typically, extrachromosomal DNA is maintained within the vector on which it was introduced into the host cell. In many instances, it may be beneficial to select a high copy number vector in order to maximize the expression of the enzyme. Multiple vectors may be present in a single cell, and in such cases vectors with compatible origins of replication are used. Optionally, the vector may further contain a selectable marker. Certain selectable markers may be used to confirm that the vector is present within the target cell. Other selectable markers may be used to further confirm that the vector and/or transgene has integrated into the host cell chromosomal DNA. The use of selectable markers is common in the art and the skilled person will understand and appreciate the many uses of selectable markers.

Whether a cell expresses or overexpresses an enzyme of the pathways described herein can easily be determined by a skilled person using a basic in vitro or in vivo enzyme assay. Common methods for measuring the amount of the product of a reaction catalysed by an enzyme may include, without limitation, chromatographic techniques such as size exclusion chromatography, separation based on charge or hydrophobicity, ion exchange chromatography, affinity chromatography, or liquid chromatography. The genetically engineered cell will yield a greater activity than a wild-type cell in such an assay. Additionally, or alternatively, the amount of an enzyme can be quantified and compared by obtaining protein extracts from the genetically engineered cell and a comparable wild-type cell and subjecting the extracts to any of number of protein quantification techniques which are well known in the art. Methods of protein quantification may include, without limitation, spectrophotometric methods, SDS-PAGE, western blotting, and mass spectrometry.

The engineered cell described herein expresses or overexpresses a methylglyoxal synthase. The host cell may or may not express a methylglyoxal synthase endogenously. If it does not express an endogenous methylglyoxal synthase, it is genetically engineered to express a methylglyoxal synthase. In one embodiment, the methylglyoxal synthase is overexpressed; i.e., the genetically engineered cell expresses a methylglyoxal synthase at a level higher than the level of methylglyoxal synthase in a comparable wild-type cell. Where a cell does not express a methylglyoxal synthase endogenously, any expression of the methylglyoxal synthase is considered to be “overexpression.” Determination of whether a methylglyoxal synthase is expressed or overexpressed can easily be made by a skilled person using a basic in vitro or in vivo enzyme assays. An exemplary in vitro methylglyoxal synthase assay is described in Example 1. Briefly, methylglyoxal synthase activity can be measured and compared by obtaining crude enzyme extracts from an engineered cell and a comparable wild-type cell, subjecting a suitable substrate (e.g., dihydroxyacetone-phosphate) to each enzyme extract, and measuring the amount of product (methylglyoxal).

A coding region encoding a methylglyoxal synthase may be obtained from a suitable biological source, such as a bacterial cell, using standard molecular cloning techniques. For example, coding regions may be isolated using polymerase chain reaction (PCR) with primers designed by standard primer design software which is commonly used in the art. Exemplary primers for use in isolating methylglyoxal synthase coding regions from C. acetobutylicum, B. subtilis, C. difficile, E. coli, T. thermophilus, K. pneumoniae, P. fluorescens, and R. eutropha can be found in Table 5. The cloned sequences are easily ligated into any standard expression vector by the skilled person.

In addition to overexpressing a methylglyoxal synthase, the genetically engineered cell described herein also expresses or overexpresses an enzyme having primary alcohol dehydrogenase activity. In one embodiment, such as those embodiments where a cell produces hydroxyacetone from methylglyoxal, a primary alcohol dehydrogenase enzyme may have methylglyoxal reductase activity. In some embodiments, the genetically engineered cell may express a sufficient level of methylglyoxal reductase activity and overexpression of methylglyoxal reductase activity is not needed. In other embodiments, it is expected that the genetically engineered cell overexpresses methylglyoxal reductase activity. Such a genetically engineered cell expresses an enzyme having methylglyoxal reductase activity at a level higher than the level of methylglyoxal reductase activity in the same comparable wild-type cell. This comparison is likewise easily made by a person of skill in the art using a basic in vitro or in vivo enzyme assays. An exemplary in vitro assay for detecting an enzyme having methylglyoxal reductase activity is described in Example 1. Briefly, methylglyoxal reductase activity can be measured and compared by obtaining crude enzyme extracts from a genetically engineered cell and a comparable wild-type cell, subjecting a suitable substrate to each enzyme extract, and measuring the decrease in absorbance of NADH.

A coding region encoding a methylglyoxal reductase may be obtained from a suitable biological source, such as a bacterial cell, using standard molecular cloning techniques. For example, coding regions may be isolated using PCR with primers designed by standard primer design software which is commonly used in the art. Exemplary primers for use in isolating methylglyoxal reductase coding regions from E. coli can be found in Table 5. The cloned sequences are easily ligated into any standard expression vector by the skilled person.

An engineered cell overexpressing a methylglyoxal synthase also expresses or overexpresses an enzyme having secondary alcohol dehydrogenase activity. A secondary alcohol dehydrogenase enzyme converts methylglyoxal to lactaldehyde and/or converts hydroxyacetone to 1,2-propanediol. In one embodiment, a secondary alcohol dehydrogenase enzyme catalyses both reactions, i.e., it converts methylglyoxal to lactaldehyde and also converts hydroxyacetone to 1,2-propanediol. In some embodiments, the genetically engineered cell may express a sufficient level of a secondary alcohol dehydrogenase, and overexpression of secondary alcohol dehydrogenase activity is not needed. In other embodiments, it is expected that the genetically engineered cell overexpresses secondary alcohol dehydrogenase activity. Such a genetically engineered cell expresses an enzyme having secondary alcohol dehydrogenase activity at a level higher than the level of the secondary alcohol dehydrogenase activity in the same comparable wild-type cell. This comparison is likewise easily made by a person of skill in the art using a basic in vitro or in vivo enzyme assays. An exemplary in vitro assay for detecting an enzyme having secondary alcohol dehydrogenase activity is described in Example 1. Briefly, secondary alcohol dehydrogenase activity can be measured and compared by obtaining crude enzyme extracts from a genetically engineered cell and a comparable wild-type cell, subjecting a suitable substrate to each enzyme extract, and measuring the decrease in absorbance of NADH.

A coding region encoding a secondary alcohol dehydrogenase may be obtained from a suitable biological source, such as a bacterial cell, using standard molecular cloning techniques. For example, coding regions may be isolated using PCR with primers designed by standard primer design software which is commonly used in the art. Exemplary primers for use in isolating secondary alcohol dehydrogenase coding regions from E. coli and K. pneumoniae can be found in Table 5. The cloned sequences are easily ligated into any standard expression vector by the skilled person.

In one embodiment, such as those embodiments where a cell produces lactaldehyde from methylglyoxal, a primary alcohol dehydrogenase enzyme may have lactaldehyde reductase activity. In some embodiments, the genetically engineered cell may express a sufficient level of lactaldehyde reductase activity, and overexpression of lactaldehyde reductase activity is not needed. In other embodiments, it is expected that the genetically engineered cell overexpresses lactaldehyde reductase activity. Such a genetically engineered cell expresses an enzyme having lactaldehyde reductase activity at a level higher than the level of lactaldehyde reductase activity in the same comparable wild-type cell. This comparison is likewise easily made by a skilled person using in vitro or in vivo enzyme assays. Such assays are known in the art and are routine. A coding region encoding a lactaldehyde reductase may be obtained from a suitable biological source, such as a bacterial cell, using standard molecular cloning techniques. The cloned sequences are easily ligated into any standard expression vector by the skilled person.

The engineered cell described herein expresses or overexpresses a diol dehydratase. The host cell may or may not express a diol dehydratase endogenously. If it does not express an endogenous diol dehydratase, it is genetically engineered to express a diol dehydratase. In one embodiment, the diol dehydratase is overexpressed; i.e., the genetically engineered cell expresses a diol dehydratase at a level higher than the level of diol dehydratase in a comparable wild-type cell. Where a cell does not express a diol dehydratase endogenously, any expression of the diol dehydratase is considered to be “overexpression.” Determination of whether a diol dehydratase is expressed or overexpressed can easily be made by a skilled person using an in vitro or in vivo enzyme assay. An exemplary in vitro diol dehydratase synthase assay is described in Example 1. Briefly, diol dehydratase activity can be measured and compared by obtaining crude enzyme extracts from an engineered cell and a comparable wild-type cell, subjecting a suitable substrate (e.g., 1,2-propanediol) to each enzyme extract, and measuring the amount of product (1-propanal). The genetically engineered cell will yield a greater activity than a wild-type cell in such an assay.

A coding region encoding a diol dehydratase may be obtained from a suitable biological source, such as a bacterial cell, using standard molecular cloning techniques. For example, coding regions may be isolated using PCR with primers designed by standard primer design software which is commonly used in the art. Exemplary primers for use in isolating diol dehydratase coding regions from K. oxytoca, K. pneumoniae, and C. butyricum are disclosed in Table 5. The cloned sequences are easily ligated into any standard expression vector by the skilled person.

An engineered cell also expresses or overexpresses an enzyme having 1-propanol reductase activity. In some embodiments, the genetically engineered cell may express a sufficient level of 1-propanol reductase activity, and overexpression of 1-propanol reductase activity is not needed. In other embodiments, it is expected that the genetically engineered cell overexpresses 1-propanol reductase activity. Such a genetically engineered cell expresses an enzyme having 1-propanol reductase activity at a level higher than the level of 1-propanol reductase activity in the same comparable wild-type cell. This comparison is likewise easily made by a skilled person using in vitro or in vivo enzyme assays. Such assays are known in the art and are routine. A coding region encoding a 1-propanol reductase activity may be obtained from a suitable biological source, such as a bacterial cell, using standard molecular cloning techniques. The cloned sequences are easily ligated into any standard expression vector by the skilled person.

A host cell may be further engineered to include other modifications. In one embodiment, modifications may include those that divert carbon flux towards 1-propanol formation. In one embodiment, the ability of a host cell to catalyse the conversion of the precursor dihydroxyacetone-phosphate to glyceraldehyde 3-phosphate can be decreased by engineering a knockout of a tpiA gene encoding triose phosphate isomerase. As used herein, the term “knockout” refers to any modification of a coding region or an operably linked regulatory region that results in decreased activity of the polypeptide encoded by the coding region. Thus, deletion of an entire coding region or a portion of a coding region, a modification of a coding region so that it encodes a polypeptide with decreased activity, and the like are included within the term “knockout.”

Some host cells metabolize glucose through the Entner-Doudoroff pathway to generate glyceraldehyde 3-phosphate at the expense of the precursor dihydroxyacetone-phosphate. This pathway to glyceraldehyde 3-phosphate is made up of four enzymes encoded by the genes zwf, pgl, edd, and eda. In one embodiment, the diversion of carbon through the Entner-Doudoroff pathway may be reduced by engineering a knockout of zwf, pgl, edd, eda, or a combination thereof.

Lactate has been observed as a by-product of the novel pathway described herein. In one embodiment, a host cell is engineered to reduce lactate production by knockout of a lactate dehydrogenase gene ldhA. Lactate can also be generated from methylglyoxal in some host cells by a native detoxifying mechanism, the glyoxalase system, which catalyzes two sequential reactions for the conversion of methylglyoxal to lactate (FIG. 6). In one embodiment, the flux of carbon away from the pathway described herein can be reduced by knockout of the enzyme encoded by gloA and/or the enzyme encoded by gloB. Carbon flux to other fermentative products such as ethanol and succinate may occur by action of the enzyme encoded by adhE, the enzyme encoded byfrd, or the combination thereof.

Methods for engineering a knockout of a coding region are known and routine. For instance, modifying a host cell to include a knockout may be carried out with, for instance, the lamda Red recombinase/FLP system or P1 transduction of the Keio collection (Baba et al., Mol. Syst. Biol. 2006, 2:2006 0008; Datsenko and Wanner, Proc. Natl. Acad. Sci. USA 2000, 97:6640-6645).

In one embodiment, a host cell is engineered to increase its ability reduce substrate. In one embodiment, a host cell is engineered to extract NADH from formate. For instance, the host cell can be engineered to express a formate dehydrogenase. A non-limiting example of a suitable coding region encoding a formate dehydrogenase is the fdh gene from Candida boidinii.

Also provided herein are methods for producing 1-propanol using the genetically engineered cell described herein. Briefly, and as described and illustrated in more detail elsewhere herein, the host cell is engineered to contain a novel biosynthetic pathway. Specifically, the host cell is engineered to overexpress an enzyme having methylglyoxal synthase activity, primary alcohol dehydrogenase activity (such as methylglyoxal reductase, lactaldehyde reductase, and/or 1-propanol reductase), secondary alcohol dehydrogenase activity (such as glycerol dehydrogenase and/or diol dehydrogenase), diol dehydratase activity, or a combination thereof. Thus, the methods provided herein provide for the synthesis of 1-propanal from the precursor dihydroxyacetone-phosphate by means of at least one, and preferably two, parallel pathways. In one embodiment, the method includes the use of two engineered cells, where one engineered cell produces the precursor 1,2-propanol, and a second engineered cell uses the 1,2-propanol and converts it to 1-propanol. In one embodiment, the amount of 1-propanol produced by an engineered cell described herein after 24 hours incubation in a shake flask is at least 0.1 gram/liter (g/L), at least 0.25 g/L, at least 0.5 g/L, at least 0.75 g/L, at least 1 g/L, at least 1.25 g/L, at least 1.5 g/L, at least 1.75 g/L, or at least 2 g/L.

The 1-propanal produced via the novel biosynthetic pathway can be isolated and optionally purified from any genetically engineered cell described herein. It can be isolated directly from the cells, or from the culture medium, for example, during an aerobic or anaerobic fermentation process. Isolation and/or purification can be accomplished using known and routine methods. The 1-propanol may be used in any application, including the biofuel industry, manufacture of drugs, cosmetics, flexographic printing ink, and textiles.

The genetically engineered cells described herein can be cultured aerobically or anaerobically, or in a multiple phase fermentation that makes use of periods of anaerobic and aerobic fermentation. The decision on whether to use anaerobic and aerobic fermentation depends on variable familiar to the skilled person. In one embodiment, for instance when the engineered cell includes enzymes of the pathway that have greater activity in anaerobic condition (e.g., certain the diol dehydratases, such as the glycerol dehydratase (GLD) from C. butyricum), the engineered cell is incubated in anaerobic conditions. Fed-batch fermentation, batch fermentation, continuous fermentation, or any other fermentation method may be used.

In various embodiments different supplements may be included in the medium in which the engineered cells are grown. For instance, depending upon the enzymes expressed in the host cell, the medium may include low levels of phosphate, one or more co-enzymes such as vitamin B12, formate, and the like. The method may also include supplying at least one carbon source such as glucose, xylose, sucrose, arabinose, and galactose.

Importantly, the present invention permits a “total synthesis” or “de novo” biosynthesis of 1-propanol in the genetically engineered cell. In other words, it is not necessary to supply the genetically engineered cells with precursors or intermediates; 1-propanol can be produced using ordinary inexpensive carbon sources such as glucose and the like.

The present invention is illustrated by the following examples. It is to be understood that the particular examples, materials, amounts, and procedures are to be interpreted broadly in accordance with the scope and spirit of the invention as set forth herein.

### Example 1

### Dehydratase Mediated 1-Propanol Production in Metabolically Engineered Escherichia coli

With the increasing consumption of fossil fuels, the question of meeting the global energy demand is of great importance in the near future. As an effective solution, production of higher alcohols from renewable sources by microorganisms has been proposed to address both energy crisis and environmental concerns. Higher alcohols contain more than two carbon atoms and have better physiochemical properties than ethanol as fuel substitutes.

We designed a novel 1-propanol metabolic pathway by expanding the well-known 1,2-propanediol pathway with two more enzymatic steps catalyzed by a 1,2-propanediol dehydratase and an alcohol dehydrogenase. In order to engineer the pathway into E. coli, we evaluated the activities of eight different methylglyoxal synthases which play crucial roles in shunting carbon flux from glycolysis towards 1-propanol biosynthesis, as well as two secondary alcohol dehydrogenases of different origins that reduce both methylglyoxal and hydroxyacetone. It is evident from our results that the most active enzymes are the methylglyoxal synthase from Bacillus subtilis and the secondary alcohol dehydrogenase from Klebsiella pneumoniae, encoded by mgsA and budC respectively. With the expression of these two genes and the E. coli ydjG encoding methylglyoxal reductase, we achieved the production of 1,2-propanediol at 0.8 g/L in shake flask experiments. We then characterized the catalytic efficiency of three different diol dehydratases on 1,2-propanediol and identified the optimal one as the 1,2-propanediol dehydratase from Klebsiella oxytoca, encoded by the operon ppdABC. Co-expressing this enzyme with the above 1,2-propanediol pathway in wild type E. coli resulted in the production of 1-propanol at a titer of 0.25 g/L.

We have successfully established a new pathway for 1-propanol production by shunting the carbon flux from glycolysis. To our knowledge, it is the first time that this pathway has been utilized to produce 1-propanol in E. coli. The work presented here forms a basis for further improvement in production. We speculate that dragging more carbon flux towards methylglyoxal by manipulating glycolytic pathway and eliminating competing pathways such as lactate generation can further enhance the production of 1-propanol.

### Results and Discussion

### Methylglyoxal Synthase Assay

1,2-Propanediol pathway branches from glycolysis and competes for the intermediate dihydroxyacetone phosphate, with the glycolytic pathway. The first enzyme of 1,2-propanediol pathway, methylglyoxal synthase catalyzing irreversible conversion of dihydroxyacetone-phosphate to methylglyoxal holds paramount importance in channeling carbon flux towards 1,2-propanediol biosynthesis (Altras and Cameron, Appl. Environ. Microbiol. 1999, 65:1180-1185; Berrios-Rivera et al., J. Indust. Microbiol. Biotech. 2003, 30:34-40). Highly active methylglyoxal synthase is therefore desirable. We screened the activity of methylglyoxal synthase from eight different sources. We amplified the mgsA genes from the microorganisms: C. acetobutylicum (ATCC#824), B. subtilis 168, C. difficile R20291, E. coli MG1655, T. thermophilus HB27, K. pneumoniae MGH78578, P. fluorescens Pf-5, and R. eutropha H16 respectively. These genes were cloned and expressed in wild type E. coli BW25113 using eight plasmids pRJ1-pRJ8. Each gene was under the control of the IPTG-inducible pLlacO1 promoter. Using dihydroxyacetone-phosphate as the substrate, we successfully detected the functional expression of all mgsA genes in vitro, where the specific activities varied from 0.0052 U/mg to 0.1242 U/mg (Table 1). We identified most suitable methylglyoxal synthase as the mgsA from B. subtilis demonstrating the highest ratio of specific activity/Kₘ (0.1186) and having a specific activity of 0.0561 U/mg. Without the over-expression of mgsA gene, we also detected the native expression of E. coli mgsA, which gave a specific activity of only 0.0008 U/mg, much lower than that of any over-expression.

TABLE 1
Methylglyoxal synthase assay results.

| | | | Specific |
|---|---|---|---|
| | Specific | | Activity/Km |
| mgsA source | Activity (U/mg) | Km (mM) | (U/mg/mM) |
| C. acetobutylicum | 0.0541 ± 0.0042 | 0.776 ± 0.005 | 0.0697 |
| B. subtilis | 0.0561 ± 0.0031 | 0.473 ± 0.070 | 0.1186 |
| C. difficile | 0.0597 ± 0.0039 | 1.439 ± 0.060 | 0.0415 |
| E. coli | 0.1242 ± 0.0069 | 1.418 ± 0.120 | 0.0876 |
| T. thermophilus | 0.0161 ± 0.0004 | 2.118 ± 0.070 | 0.0076 |
| K. pneumoniae | 0.0165 ± 0.0009 | 2.820 ± 0.300 | 0.0058 |
| P. fluorescens | 0.0133 ± 0.0082 | 1.560 ± 0.020 | 0.0085 |
| R. eutropha | 0.0052 ± 0.0004 | 0.700 ± 0.030 | 0.0074 |

Substrate dihydroxyacetone phosphate concentration was varied from 0.15 mM to 1.5 mM for all reactions. 1 unit (U) was defined as the amount (μmoles) of methylglyoxal formed per unit time (min).

### Methylglyoxal Reductase Assay

We examined the activity of E. coli methylglyoxal reductase encoded by the gene ydjG by using plasmid pRJ10. As a part of aldo-keto reductase family, the product of ydjG executes a catalytic activity of reduction on methylglyoxal using NADH to generate hydroxyacetone (Luccio et al., Biochem. J. 2006, 400:105-114). We determined both the specific activity and substrate affinity of E. coli methylglyoxal reductase on methylglyoxal. When the gene is over-expressed by pRJ10, the specific activity was determined to be 1.62±0.012 U/mg. The enzyme also showed sufficient substrate specificity with a Kₘ value of 3.31±0.02 mM.

### Secondary Alcohol Dehydrogenase Assay

The synthesis of 1,2-propanediol from methylglyoxal occurs through two different pathways. For the pathway via lactaldehyde leading to 1,2-propanediol formation we evaluated the activities of two NADH dependent secondary alcohol dehydrogenases: E. coli glycerol dehydrogenase (gldy-4) and K. pneumoniae diol dehydrogenase (budC) on methylglyoxal. We also tested the catalytic properties of these two secondary alcohol dehydrogenases on hydroxyacetone for the completion of the other pathway.

The genes gldA and budC were cloned and expressed in E. coli using the plasmid pRJ9 and pYY109. The specific activity and Kₘ value of glycerol dehydrogenase and diol dehydrogenase were determined for the substrates methylglyoxal and hydroxyacetone. Both enzymes showed dehydrogenation activity leading to the conversion of methylglyoxal to lactaldehyde and hydroxyacetone to 1,-2-propanediol. Table 2 provides the results of this assay. The diol dehydrogenase and glycerol dehydrogenase reduced both methylglyoxal and hydroxyacetone. When methylglyoxal was used as the substrate, the diol dehydrogenase demonstrated a specific activity of 3.718 U/mg with a Kₘ value of 0.78 mM; while the glycerol dehydrogenase showed both lower specific activity (2.456 U/mg) and substrate affinity (Kₘ=68.24 mM). Similar results were observed when hydroxyacetone was tested as a substrate, the diol dehydrogenase more efficiently reduced hydroxyacetone into 1,2-propanediol (specific activity=4.97 U/mg; Kₘ=1.83 mM) compared with the glycerol dehydrogenase (specific activity=0.912 U/mg; Kₘ=10.47 mM).

TABLE 2
Specific activity and Kₘ determination of the secondary
alcohol dehydrogenases.

| | Methylglyoxal | | Hydroxyacetone | |
|---|---|---|---|---|
| | Specific Activity | Kₘ | Specific Activity | Kₘ |
| Gene | (U/mg) | (mM) | (U/mg) | (mM) |
| gldA | 2.456 ± 0.001 | 68.24 ± 0.05 | 0.912 ± 0.008 | 10.47 ± 0.55 |
| budC | 3.718 ± 0.066 | 0.78 ± 0.03 | 4.970 ± 0.007 | 1.83 ± 0.63 |

The decrease in absorbance of NADH at 340 nm was recorded and used for calculations using the substrates methylglyoxal and hydroxyacetone. Substrate concentration was varied from 20 mM-120 mM. 1 unit (U) was defined as the amount (μmoles) of product formed per unit time (min).

### Propanediol Dehydratase In Vivo Assay

The diol dehydratases we tested included a propanediol dehydratase (PPD) originating in K. oxytoca, a glycerol dehydratase (GLD) from K. pneumoniae, and a glycerol dehydratase (GLD) from C. butyricum. The PPD of K. oxytoca and the GLD of K. pneumoniae are iso-functional enzymes which catalyze the coenzyme B12-dependent conversion of 1,2-propanediol or glycerol to the corresponding aldehyde (Luccio et al., Biochem. J. 2006, 400:105-114; Honda et al., J. Bacteriol. 1980, 143:1458-1465; Tobimatsu et al., J. Biolog. Chem. 1996, 271:22352-22357; Tobimatsu et al., Arch. Biochem. Biophys. 1997, 347:132-140). These enzymes have been utilized to develop a biological process to produce 1,3-propanediol from glycerol (Raynaud et al., Proc. Natl. Acad. Sci. USA 2003, 100:5010-5015). Each of these enzymes consists of three subunits encoded by three structural genes (ppdABC or gldABC). Although the catalytic site is hosted by subunit A, the presence of subunits B and C are obligatory for enzyme activity (Tobimatsu et al., J. Biolog. Chem. 1996, 271:22352-22357). In order to evaluate their catalytic efficiency towards 1,2-propanediol, all three subunits were co-expressed in E. coli to reconstitute the enzymes using the plasmids pYY93 and pYY134. The GLD from C. butyricum is a coenzyme B 12-independent diol dehydratase comprised of two subunits encoded by dhaB12, which only demonstrates activity under strict anaerobic conditions (Raynaud et al., Proc. Natl. Acad. Sci. USA 2003, 100:5010-5015). To evaluate its catalytic efficiency, we constructed the plasmid pYY167 to co-express these two units.

The formation of 1-propanol from 1,2-propanediol involves two enzymatic steps. For the first step we evaluated the dehydration activity of three different diol dehydratases for the generation of 1-propanal. For the second step we relied on the native alcohol dehydrogenase activity of E. coli to convert the generated 1-propanal to 1-propanol. An experiment was designed and conducted as we described to perforin the in vivo enzyme assay of propanediol dehydratase and also to evaluate the native activity of E. coli for the final step. Whole-cell bioconversion studies using wild type E. coli strain BW25113 carrying pYY93, pYY134, and pYY167 respectively were conducted in shake flasks by feeding 5 g/L (65.7 mM) 1,2-propanediol as the substrate. The samples were collected after 24 hours and analyzed by HPLC-RID.

The results are presented in FIG. 4. The catalytic efficiency of K. oxytoca PPD was the highest among all, producing 65.6 mM 1-propanol amounting to nearly 100% conversion. This result also indicated that the native expression of alcohol dehydrogenases in E. coli is sufficient to convert 1-propanal to 1-propanol completely. Over-expression of the alcohol dehydrogenases will not be necessary for 1-propanol production in E. coli. The K. pneumoniae GLD and C. butyricum GLD only demonstrated about 60.9% and 30.9% of the catalytic efficiency of K. oxytoca PPD, producing 39.99 mM and 20.35 mM 1-propanol, respectively.

Production of 1,2-Propanediol and 1-Propanol in E. coli

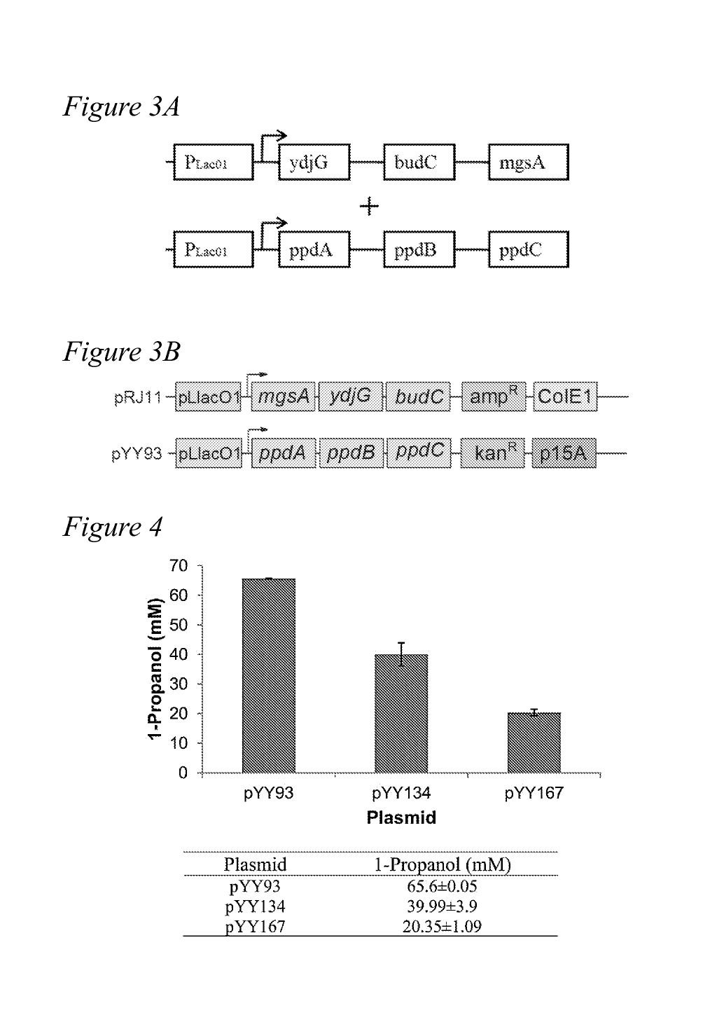

In order to introduce the 1-propanol pathway into wild type E. coli strain BW25113, we constructed two plasmids (FIG. 3). The first plasmid pRJ11 carries the genes encoding the most active enzymes for 1,2-propanediol biosynthesis. Specifically, mgsA from B. subtilis, ydjG from E. coli, and budC from K. pneumoniae were organized as a synthetic operon under the control of IPTG-inducible pLlacO1 promoter in a high-copy number plasmid. The second plasmid pYY93 contains only the structural genes ppdABC encoded by K. oxytoca PPD. For the enzymatic steps of lactaldehyde to 1,2-propanediol and 1-propanal to 1-propanol, we completely relied on the native expression of alcohol dehydrogenases in E. coli, as is indicated to be sufficient (Altras and Cameron, Appl. Environ. Microbiol. 1999, 65:1180-1185; Berrios-Rivera et al., J. Indust. Microbiol. Biotech. 2003, 30:34-40). We also evaluated M9 media in comparison to low-phosphate media for the production of 1,2-propanediol. The results of an initial experiment show significant increase in production using low-phosphate media. Using M9 media resulted in only about 0.1 g/L of 1,2-propanediol generation after 48 hours of anaerobic fermentation compared to about 0.8 g/L from low-phosphate media. Hence low-phosphate media was used for all fermentation studies. The culture conditions were the same as described in “Methods and Materials”.

We first transformed the plasmid pRJ11 into wild type E. coli strain BW25113 to achieve 1,2-propanediol production. It has been reported that the enzyme methylglyoxal synthase is inhibited by phosphate ion (Hopper and Cooper, Biochem. J. 1972, 128:321-329; Hopper and Cooper, Fed. Eur. Biochem. Soc. Lett. 1971, 13:213-216). The inhibition of methylglyoxal synthase would result in the carbon flux being diverted to glyceraldehyde-3-phosphate instead of methylglyoxal by the conversion action of triose phosphate isomerase. Therefore a low-phosphate media was employed to avoid this. The fermentation experiments were conducted in 20 ml cultures as described in the “Methods and Materials”. The fermentation samples were collected after 24 hours and 48 hours and analyzed by HPLC-RID. As the results show in Table 3, after 24 hours 0.66 g/L 1,2-propanediol was produced. The production reached 0.80 g/L after 48 hours. Lactate was detected as the dominant by-product and was accumulated at over 7 g/L. We also conducted the experiments aerobically. However, only a trace amount (<0.01 g/L) of 1,2-propanediol was produced and the cell growth was much better than in anaerobic conditions, which indicates that glycolysis is very active in aerobic condition and drags almost all carbon flux towards pyruvate for cell growth or other cell activities.

With the successful establishment of the pathways for 1,2-propanediol production using pRJ11, we hypothesized that the co-expression of pRJ11 with pYY93 would result in the production of 1-propanol. To test this, wild type E. coli BW25113 was transformed with both pRJ11 and pYY93 for 1-propanol production by electroporation. The fermentation condition was similar to that used for 1,2-propanediol production with the addition of 10 μM coenzyme B-12 to the culture along with IPTG (0.1 mM) after 6 hours. After 24 hours, the double transformed strain produced 0.11 g/L 1-propanol with 0.44 g/L 1,2-propanediol remaining unconverted (Table 3). After 48 hours, 1-propanol was produced at 0.25 g/L with 0.46 g/L 1,2-propanediol remaining unconverted. The major by-product was again lactate at over 7 g/L.

TABLE 3
1,2-Propanediol and 1-propanol production in low-phosphate
media using E. coli strain BW25113 transformed with the
appropriate plasmid(s).

| | 1,2-Propanediol (g/L) | | 1-Propanol (g/L) | |
|---|---|---|---|---|
| Plasmid | 24 h | 48 h | 24 h | 48 h |
| pRJ11 | 0.66 ± 0.01 | 0.80 ± 0.01 | — | — |
| pRJ11 and | 0.44 ± 0.01 | 0.46 ± 0.02 | 0.11 ± 0.01 | 0.25 ± 0.07 |
| pYY93 | | | | |

### CONCLUSIONS

We have successfully established a new pathway for 1-propanol production by shunting the native glycolytic pathway in E. coli. The addition of the coenzyme B-12 dependent propanediol dehydratase from K. oxytoca resulted in the conversion of 1,2-propanediol to 1-propanal which was then dehydrogenated by E. coli native activity to 1-propanol.

From the assay of methylglyoxal synthase it was determined that the mgsA from B. subtilis was the most active. Since the accumulation of methylglyoxal in high quantities is toxic to the cell (Ackerman et al., Journal of Bacteriology 1974, 119:357-352), it is important that the generated methylglyoxal is immediately converted to another metabolite by the downstream enzymes in the pathway. To address this we screened the activity of methylglyoxal reductase and two secondary alcohol dehydrogenases.

To evaluate 1,2-propanediol formation, methylglyoxal synthase (mgsA) from B. subtilis and propanediol dehydratase (budC) from K. pneumoniae were expressed leading to the conversion of dihydroxyacetone phosphate to 1,2-propanediol via the formation of methylglyoxal and lactaldehyde. To strengthen our constructed pathway the introduction of E. coli methylglyoxal reductase (ydjG), a dual metabolic route for production of 1,2-propanediol was established for the first time. This resulted in channeling the carbon flux from methylglyoxal to hydroxyacetone and 1,2-propanediol. Fermentation with E. coli BW25113 transformed with pRJ11 carrying the three above mentioned genes produced 0.8 g/L 1,2-propanediol after 48 hours of anaerobic fermentation.

We also evaluated the use of a medium copy number vector (pRJ12) for 1,2-propanediol production. This was done using the same genes used for the construction of high copy number plasmid pRJ11 but in the backbone of a medium copy number vector pCS27. However, the production of 1,2-propanediol from a medium copy number vector (pRJ12) was found to be significantly lower than the production by high copy number vector (pRJ11). Hence the medium copy number vector was not selected for 1,2-propanediol and 1-propanol production.

The result of the in vivo enzyme assay (FIG. 4) shows almost 100% conversion of 1,2-propanediol to 1-propanol indicating that the conversion of 1,2-propanediol to 1-propanal was very efficient and that the native expression of alcohol dehydrogenases in E. coli is sufficient in converting 1-propanal to 1-propanol. However, it was not the case for the strain carrying the plasmids pRJ11 and pYY93 which showed much lower conversion of 1,2-propanediol to 1-propanol as about 0.46 g/L of 1,2-propanediol was left unconverted. We speculate that the reason for this could be the expression issue of ppdABC. The optimal expression of these three subunits can be successfully achieved in aerobic conditions as we did in in vivo assay (Tobimatsu et al., J. Biolog. Chem. 1996, 271:22352-22357). However, in anaerobic conditions which is required for 1,2-propanediol production, the protein expression might be negatively affected due to low cellular energy and nutrients. This can be resolved in a more controlled environment such as in a bench scale fermenter by the delicate adjustment of oxygen level during fermentation course.

The accumulation of 7 g/L lactate indicates that the carbon flux towards pyruvate is still strong in anaerobic conditions. The main branch of glycolysis plays the major role. Theoretically, one molecule of fructose-1,6-bisphosphate is broken down into one molecule of glyceraldehyde-3-phosphate and one molecule of dihydroxyacetone phosphate (Stribling and Perham, Biochem. J. 11973, 131:833-841). However, the presence of triose phosphate isomerase seems to channel the carbon flux back to the main branch toward pyruvate biosynthesis (Stribling and Perham, Biochem. J. 1973, 131:833-841). In addition, the pentose phosphate pathway is also very active in low phosphate conditions (Kruger and Schaewen, Curr. Opin. Plant Biol. 2003, 6:236-246). This pathway does not generate dihydroxyacetone phosphate as an intermediate, but directly goes to pyruvate. The pyruvate generated is acted upon by lactate dehydrogenase (ldhA) resulting in the production of lactate (Jiang et al., Microbiol. 2001, 147:2437-2466). Another minor route of lactate formation is via the glyoxalase pathway where methylglyoxal is converted to lactate by the native expression of gloA (Clugston et al., Biochem. J. 1998, 37(24):8754-63).

Overall, the work presented here represents 1-propanol production in a wild type E. coli strain and forms a basis for further enhancement in production. The effect of competing pathways is significant and the deletion of the same has not been explored in this study. We speculate that by the knock-out of genes encoding for lactate dehydrogenase (ldhA), glyoxalaseI (gloA) and other competing pathways (tpiA and zwf) the production of 1-propanol can be further enhanced, which will be pursued in the near future.

### Materials and Methods

### Chemicals and Reagents

Hydroxyacetone was bought from Acros Organics (New Jersey, USA); methylglyoxal and 1,2-propanediol were purchased from Sigma Aldrich (St. Louis, Mo.); 1-propanol was obtained from Fisher Scientific (Atlanta, Ga.). KOD DNA polymerase was obtained from EMD Chemicals Inc., NJ. All restriction enzymes were bought from New England Biolabs (Beverly, Mass.). The rapid DNA ligase was obtained from Roche Applied Science (Indianapolis, Ind.). All the enzymes were used according to the instructions of the manufacturer.

### Plasmids and Strains

E. coli strain XL1-Blue (Stratagene, CA) was used for DNA manipulations; while wild type E. coli strain BW25113 (E. coli Genetic Resource Center, CT) and E. coli strain BL21* (Invitrogen) were employed for enzyme assays and shake flask experiments. Plasmids pZE12-luc (Lutz and Bujard, Nucleic Acids Research 1997, 25:1203-1210), pCS27 (Shen and Liao, Metabolic Engineering 2008, 10:312-320) and pCDF-Duet1 (EMD Chemicals Inc., NJ) were used for DNA cloning. The features and descriptions of the used strains and plasmids are listed in Table 4.

TABLE 4
List of strains and plasmids used in this study.

| Strain | Genotype | Reference |
|---|---|---|
| E. coli | rrnBT14 DlacZWJ16 hsdR514 DaraBADAH33 | E. coli Genetic Resources at Yale |
| BW25113 | DrhaBADLD78 | CGSC, The Coli Genetic Stock |
| | | Center |
| E. coli | F⁻ ompT hsdS_{B} (r_{B}⁻ m_{B}⁻) gal dcm (DE3) | Invitrogen |
| BL21* | | |
| E. coli XL-1 | recA1 endA1gyrA96thi-1hsdR17supE44relA1lac | Stratagene |
| Blue | [F′ proAB lacIqZDM15Tn10 (TetR)] | |
| Plasmid | Description | Reference |
| pZE12-luc | pLlacO1::luc(VF); ColE1 ori; Amp^{R} | Lutz and Bujard, Nucleic Acids |
| | | Research 1997, 25: 1203-1210 |
| pCS27 | pLlacO1:: MCS; p15A ori; Kan^{R} | Shen and Liao, Metabolic |
| | | Engineering 2008, 10:312-320 |
| pCDF-Duet1 | pT7lac::MCS; CDF ori; Sm^{R} | EMD Chemicals Inc., NJ |
| pYY93 | ppdABC from K. oxytoca cloned into pCS27 | This study |
| pYY109 | budC from K. pneumoniae cloned into pCDF- | This study |
| | Duet1 | |
| pYY134 | gldABC from K. pneumoniae cloned into pCS27 | This study |
| pYY167 | dhab12 from C. butyricum cloned into pCS27 | This study |
| pRJ1 | mgsA from C. acetobutylicum cloned into pZE12- | This study |
| | luc | |
| pRJ2 | mgsA from B. subtilis cloned into pZE12-luc | This study |
| pRJ3 | mgsA from C. difficile cloned into pZE12-luc | This study |
| pRJ4 | mgsA from E. coli cloned into pZE12-luc | This study |
| pRJ5 | mgsA from T. thermophilus cloned into pZE12- | This study |
| | luc | |
| pRJ6 | mgsA from K. pneumoniae cloned into pZE12-luc | This study |
| pRJ7 | mgsA from P. fluorescens cloned into pZE12-luc | This study |
| pRJ8 | mgsA from R. eutropha cloned into pZE12-luc | This study |
| pRJ9 | gldA from E. coli cloned into pZE12-luc | This study |
| pRJ10 | ydjG from E. coli cloned into pZE12-luc | This study |
| pRJ11 | ydjG from E. coli, budC from K. pneumoniae, and | This study |
| | mgsA | |
| | from B. subtilis cloned in pZE12-luc | |

TABLE 5
Primers used in this study.

| Plasmid | Gene | Primer Sequence (5′-3′) | SEQ ID NO: |
|---|---|---|---|
| pRJ1 | mgsA | F: GGGAAAGGTACCATGGCACTTATAATGAATAGTAAAAAAAAGATAGC | 13 |
| | | R: GGGAAAGCATGCTTAAAAATTGTCTTTTCTAATTTTTTGGTAATAAT | 14 |
| pRJ2 | mgsA | F: GGGAAAGGTACCATGAAAATTGCTTTGATCGCGCATG | 15 |
| | | R: GGGAAAGCATGCTTATACATTCGGCTCTTCTCCCCGA | 16 |
| pRJ3 | mgsA | F: GGGAAAGGTACCATGAATATAGCATTAGTAGCACATGACCAAATGAA | 17 |
| | | R: GGGAAAGCATGCTTAAATACGTTGACTTTTGCTTTTTCTAACTTCTC | 18 |
| pRJ4 | mgsA | F: GGGAAAGGTACCATGGAACTGACGACTCGCAC | 19 |
| | | R: GGGAAAGCATGCTTACTTCAGACGGTCCGCGA | 20 |
| pRJ5 | mgsA | F: GGGAAAGGTACCATGCCCATGAAGGCCCTGGC | 21 |
| | | R: GGGAAAGCATGCCTATTGGGGGGTTCCCTTGC | 22 |
| pRJ6 | mgsA | F: GGGAAAGGTACCATGTGGAATGAAAATATGGAACTGACAACACGTAC | 23 |
| | | R: GGGAAAGCATGCTTATTTCAGGCGCTCGGCAA | 24 |
| pRJ7 | mgsA | F: GGGAAATGTACAATGATCGGTATCAGTTTCACCC | 25 |
| | | R: GGGAAAGCATGCTTATCCTCGGCCGGCCAGGTA | 26 |
| pRJ8 | mgsA | F: GGGAAAGGTACCATGACTCGCCCCCGCATCGCGTTGAT | 27 |
| | | R: GGGAAATCTAGATCAGCTGGCCGCCGCTTCGT | 28 |
| pRJ9 | gldA | F: GGGAAAGCATGCAGGAGATATACCATGGACCGCATTATTCAATCACCGG | 29 |
| | | R: GGGAAATCTAGATTATTCCCACTCTTGCAGGAAACGC | 30 |
| pRJ10 | ydjG | F: GGGAAAGGTACCATGAAAAAGATACCTTTAGGCACAACGG | 31 |
| | | R: GGGAAATCTAGATTAACGCTCCAGGGCCTCTGCCATTTCC | 32 |
| pRJ11 | ydjG | F: GGGAAAGGTACCATGAAAAAGATACCTTTAGGCACAACGG | 33 |
| | budC | R: GGGAAAGTCGACTTAACGCTCCAGGGCCTCTGCCATT | 34 |
| | mgsA | F: GGGAAAGTCGACAGGAGATATACCATGAAAAAAGTCGCACTTGTTACCGG | 35 |
| | | R: GGGAAACTGCAGTTAGTTAAACACCATCCCGCCGTCG | 36 |
| | | F: GGGAAACTGCAGAGGAGATATACCATGAAAATTGCTTTGATCGCGCATGAC | 37 |
| | | R: GGGAAATCTAGATTATACATTCGGCTCTTCTCCCCGA | 38 |
| pYY93 | ppdABC | F: GGGAAACGTACGATGAGATCGAAAAGATTTGAAGCACTGGCGAAACG | 39 |
| | | R: GGGAAAAAGCTTTTAATCGTCGCCTTTGAGTTTTTTACGCTCGACG | 40 |
| pYY109 | budC | F: GGGAAAGGATCCGAAAAAAGTCGCACTTGTTACCGGCG | 41 |
| | | R: GGGAAAGTCGACTTAGTTAAACACCATCCCGCCGTCG | 42 |
| pYYl34 | gldABC | F: GGGCCCGGTACCATGAAAAGATCAAAACGATTTGCAGTACTGGCCCA | 43 |
| | | R: GGGCCCAAGCTTTTAGCTTCCTTTACGCAGCTTATGCCGCTGCTGAT | 44 |
| pYY167 | dhaB12 | F: GGGAAAGGTACCATGATCAGCAAAGGGTTCAGCACCCAG | 45 |
| | | R: GGGAAAAAGCTTTTATTCCGCGCCTATAGTACACGGAATGCCCATAA | 46 |

Bold nucleotides represent restriction sites.
Italicized nucleotides represent ribosome binding sites inserted in the primer.

### DNA Manipulations

All DNA manipulations were performed according to the standard procedures as described previously (Ausubel et al., (Eds.) Current Protocols in Molecular Biology. New York, N.Y.: John Wiley & Sons; 1994). The primers involved in DNA manipulations are listed in Table 5. The plasmids listed in Table 4 were constructed as described below.

For the methylglyoxal synthase assay, the plasmids pRJ1-pRJ8 were constructed by cloning mgsA genes from eight different sources into the vector pZE12-luc separately. Using the primers listed in Table 5, the mgsA genes were PCR amplified from the genomic DNA of C. acetobutylicum (ATCC824), B. subtilis 168, Clostridium difficile R20291, E. coli MG1655, Thermus thermophilus HB27, K. pneumoniae MGH78578, Pseudomonas fluorescens Pf-5, and Ralstonia eutropha H16 respectively. The DNA fragments obtained were digested with restriction enzymes for three hours. Acc65I and SphI restriction enzymes were used to digest mgsA genes from C. acetobutylicum (ATCC824), B. subtilis 168, C. difficile R20291, E. coli MG1655, T. thermophilus HB27, K. pneumoniae MGH78578, BsrGI and SphI for the mgsA from P. fluorescens Pf-5, and Acc65I and XbaI for the mgsA from R. eutropha H16. The vector pZE12-luc was also digested with the appropriate restriction enzymes for the above mentioned eight genes. The digested genes were then inserted into the vector pZE12-luc separately.

In order to determine the activity of methylglyoxal reductase, the plasmid pRJ10 was constructed. The ydjG gene PCR amplified from E. coli MG1655 was cloned into pZE-12luc with restriction enzymes Acc65I and XbaI generating pRJ10. For the assay of secondary alcohol dehydrogenases, plasmids pRJ9 and pYY109 were constructed. The gldA gene from E. coli MG1655 was inserted into pZE12-luc vector using restriction enzymes SphI and XbaI for the construction of plasmid pRJ9. We created pYY109 by inserting the budC gene from K. pneumoniae MGH78578 into pCDF-Duet1 vector. The restriction enzymes used for the construction of plasmid pYY109 were BamHI and SalI.

For diol dehydratase assay, plasmids pYY93, pYY134, and pYY167 were constructed. The ppdABC operon obtained via PCR from the genomic DNA of K. oxytoca was digested with restriction enzymes BsiWI and HindIII and inserted into plasmid pCS27 digested by BsiWI and HindIII. Similarly, the gldABC operon was PCR amplified from genomic DNA of K. pnuemoniae MGH 78578 and inserted into pCS27 using restriction enzymes Acc65I and HindIII. To construct pYY167, we first synthesized the operon dhaB12 from Clostridium butyricum by a PCR assembly of 50 by oligonucleotides designed from Helix Systems (NIH). The codons were optimized for E. coli standard expression. The operon was cloned into pCS27 using Acc65I and HindIII, forming pYY167.

Following the enzyme assays, plasmid pRJ11 was constructed using the most active enzymes in order to produce 1,2-propanediol. The plasmid pRJ11 was generated via the ligation of three genes on the backbone of pZE12-luc vector. The genes ydjG, budC and mgsA were PCR amplified using the primers listed in Table 5 from the genomic DNA of E. coli MG1655, K. pnuemoniae MGH78578 and B. subtilis 168, respectively. Following this, the PCR amplified ydjG gene product was digested with Acc65I and SalI. The PCR amplified budC gene product was digested with SalI and PstI, and the PCR amplified mgsA gene was digested with PstI and XbaI. Vector pZE12-luc was digested with Acc65I and XbaI. After digestion for three hours, the three gene fragments and the vector were ligated simultaneously, creating pRJ11. It should be noted that RBS sequence (AGGAGA; SEQ ID NO:47) was inserted upstream of each structure gene with 6-8 nucleotides in between to facilitate protein translations.

### Culture Medium and Fermentation Conditions

M9 minimum was used for the in-vivo assay of propanediol dehydratase and low-phosphate minimum medium was used for shake flask fermentations. M9 minimum media consisted of (per liter): 20 g glucose, 5 g yeast extract, 12.8 g Na₂HPO₄.7H₂O, 3 g KH₂PO₄, 0.5 g NaCl, 1 g NH₄Cl, 0.5 mM MgSO₄, and 0.05 mM CaCl₂. The low-phosphate media consisted of (per liter): 20 g glucose, 5 g NaCl, 5 g yeast extract, 1.5 g KCl, 1 g NH₄Cl, 0.2 g MgCl₂, 0.07 g Na₂SO₄, and 0.005 g FeCl₃, which was buffered to pH 6.8 with 13.3 g of NaHCO₃ and 10 g of 3-[N-morpholino] propanesulfonic acid (MOPS) (Altras and Cameron, Appl. Environ. Microbiol. 1999, 65:1180-1185; Altras and Cameron, Biotech. Prog. 2000, 16:940-946).

For the shake flask fermentations, 1 mL of seed culture was prepared in LB media containing necessary antibiotics and grown overnight at 37° C. in a shaker set at 250 rpm. After overnight incubation, the culture was inoculated into 20 mL of M9 or low-phosphate media containing appropriate antibiotics in 150 mL serum bottles. After growing at 37° C. for 3 hours, the cultures were switched to an anaerobic condition by sparging nitrogen gas. IPTG was added into the culture to a final concentration of 0.1 mM 6 hours after inoculation to induce protein expression. Then the fermentation was carried out at 30° C. at 250 rpm. Samples were taken after 24 and 48 hours and analyzed with HPLC-RID.

### HPLC-RID Analysis

The analysis of fermentation products was done via HPLC (Shimadzu) equipped with a Coregel-64H column (Transgenomic). 1 mL sample was collected and centrifuged at 15,000 rpm for 10 minutes and the supernatant was filtered and used for analysis. The mobile phase used was 4 mN H₂SO₄ having a flow rate of 0.6 mL/min and an oven temperature set at 60° C. (Eiteman and Chastain, Analytica Chimica Acta 1997, 338:69-75).

### Enzyme Crude Extract Preparation

E. coli strain BL21* was employed to express budC carried by pYY109. Expression of other individual enzymes was conducted in the wild type E. coli strain BW25113 harboring the corresponding plasmids. Generally, the transformed strains were pre-inoculated into LB liquid medium containing appropriate antibiotics and grown at 37° C. overnight with shaking. The following day, 1 mL of preinoculum was added to 50 mL of fresh LB medium containing necessary antibiotics. The culture was left to grow at 37° C. with shaking until the OD₆₀₀ reached approximately 0.6. At that point, IPTG was added to a final concentration of 1 mM and the protein expression was conducted at 30° C. for 3 hours. The cells were collected by centrifugation at 5000 rpm for 10 min at 4° C. The cell pellets were resuspended in 2 mL of 50 mM imidazole-HCl buffer (pH 7.0). Cell disruption was performed using French Press, and soluble protein was obtained by ultra-centrifugation for enzyme assays. Total protein concentration was estimated using the BCA kit (Pierce Chemicals).

### Methylglyoxal Synthase Assay

Methylglyoxal synthase assay was performed as described previously with minor revisions (Altras and Cameron, Appl. Environ. Microbiol. 1999, 65:1180-1185; Berrios-Rivera et al., J. Indust. Microbiol. Biotech. 2003, 30:34-40; Hopper and Cooper, Fed. Eur. Biochem. Soc. Lett. 1971, 13:213-216). The assay was carried out using a two-step procedure. The reaction mixture (500 μl) consisted of 50 mM imidazole-HCl buffer (pH 7.0), 0.15-1.5 mM dihydroxyacetone phosphate, and 25 μl crude extract. Reaction was started with the addition of 25 μl crude extract to the reaction mixture and incubated in a water bath at 30° C. for 30 seconds. Followed by this, the reaction was immediately stopped by the addition of 30 μl sample of the reaction mixture to the detection mixture and incubated in a water bath at 30° C. for 15 minutes. The detection mixture consisted of 300 μl of DI water, 110 μl of 0.1% 2,4-dinitrophenylhydrazine dissolved in 2N HCl. After the completion of 15 minutes 550 μl of 10% NaOH was added to the detection mixture and then incubated at room temperature for another 15 minutes. (Final volume of detection mixture=990 μl). The samples were diluted 10 times before measuring the absorbance at 550 nm. 1 mmol of methylglyoxal has an absorbance value of 16.4 at 550 nm (Berrios-Rivera et al., J. Indust. Microbiol. Biotech. 2003, 30:34-40).

### Methylglyoxal Reductase Assay

The reaction mixture contained 20-120 mM methylglyoxal and 0.25 mM NADH, in imidazole-HCl buffer (pH 7.0) having a final volume of 970 μl. The assay was begun with the addition of 30 μl crude extract to the reaction mixture. The reaction was allowed to proceed for 60 seconds at 37° C. We measured the decrease in absorbance of NADH at 340 nm to calculate the specific activity (Altras and Cameron, Appl. Environ. Microbiol. 1999, 65:1180-1185; Berrios-Rivera et al., J. Indust. Microbiol. Biotech. 2003, 30:34-40).

### Secondary Alcohol Dehydrogenase Assay

The enzyme crude extracts prepared from gldA and budC expression were used for this assay. The reaction mixture consisted of 20-120 mM of methylglyoxal or hydroxyacetone and 0.25 mM NADH in imidazole-HCl buffer at (pH 7.0) with a final volume of 970 μl. The assay was begun with the addition of 30 μl crude extract to the reaction mixture. The reaction was allowed to proceed for 60 seconds at 37° C. We measured the decrease in absorbance of NADH at 340 nm to calculate the specific activity (Altras and Cameron, Appl. Environ. Microbiol. 1999, 65:1180-1185; Berrios-Rivera et al., J. Indust. Microbiol. Biotech. 2003, 30:34-40).

### Propanediol Dehydratase In Vivo Assay

The assay was carried out to evaluate the activities of three diol dehydratases on 1,2-propanediol. Three E. coli strains generated by transforming the wild type E. coli BW25113 with pYY93, pYY134, and pYY167 respectively were used for this purpose. Preinoculum from an overnight culture was added to 10 mL of M9 media (1:100 UV) and grown at 37° C. IPTG was added to the cultures to a final concentration of 0.1 mM and 1,2-propanediol was added to the cultures as the substrate to a final concentration of 5 g/L (65.7 mM) after 4 hours. The cell cultures carrying pYY167 was grown anaerobically; while the cell cultures carrying pYY93 or pYY134 were grown micro-aerobically. Coenzyme-B12 (cobamamide) was also added to the cell cultures having pYY93 and pYY134 to a final concentration of 10 μM after 4 hours. Samples were collected after 24 hours and analyzed for 1-propanol generation using HPLC-RID as described above. The enzyme activities were reflected by the formation of 1-propanol.

### Example 2

### Increasing Diol Dehydratase Expression Using Different Expression Strategies

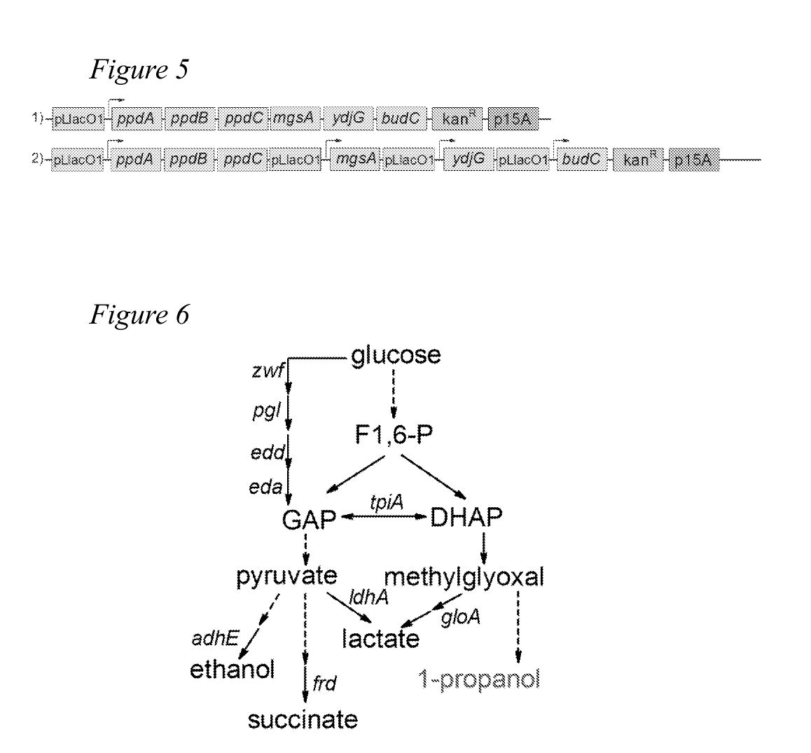

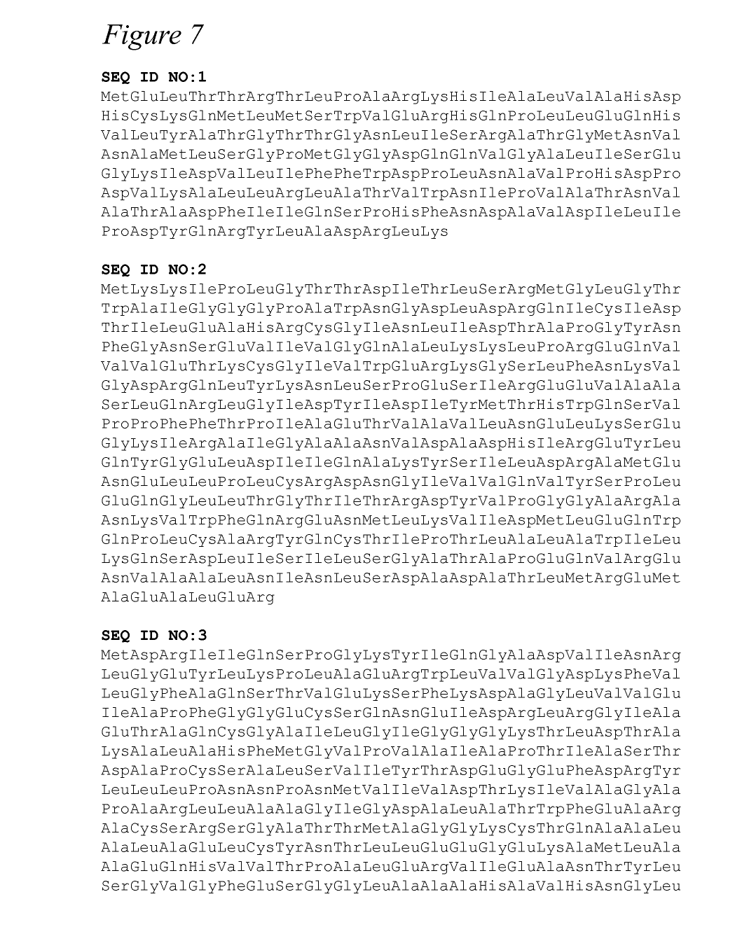

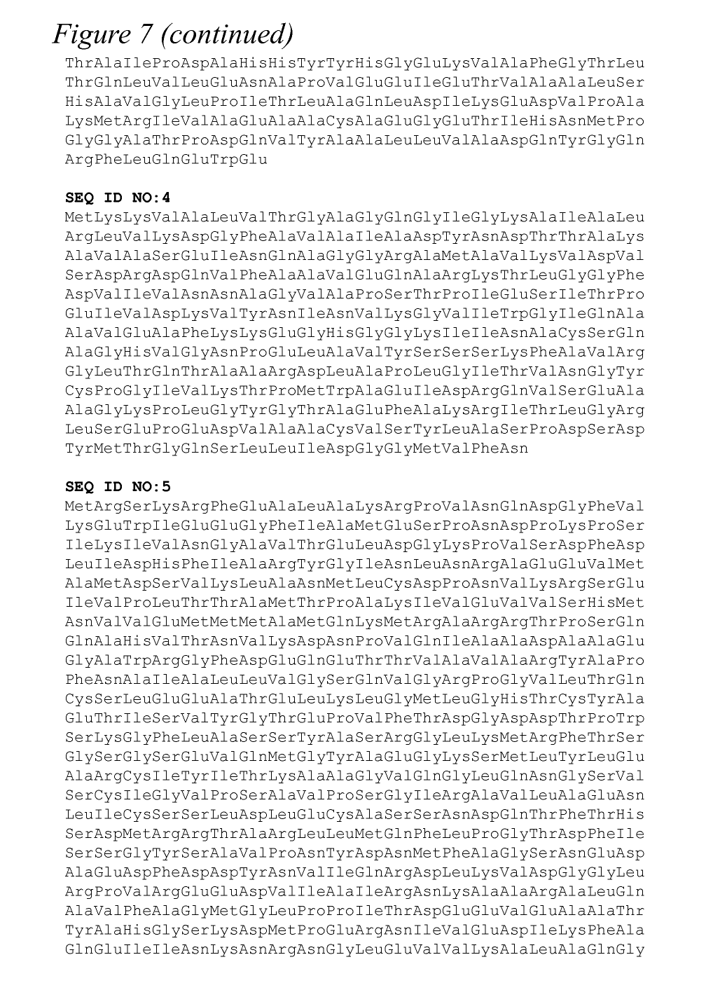

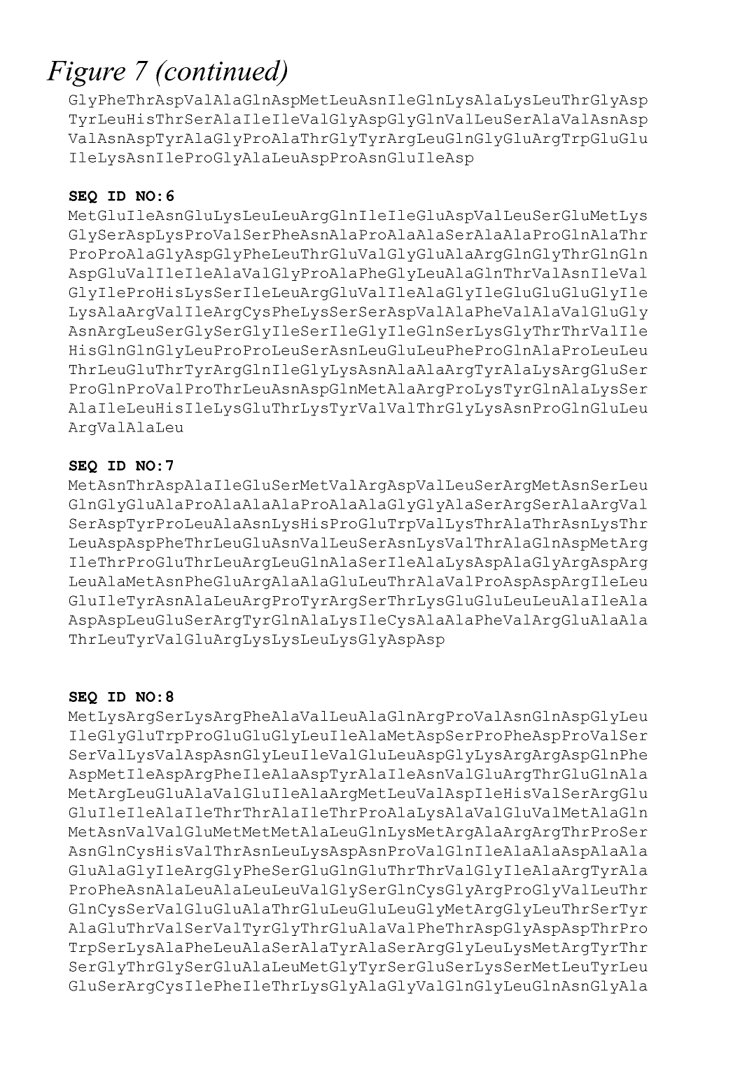

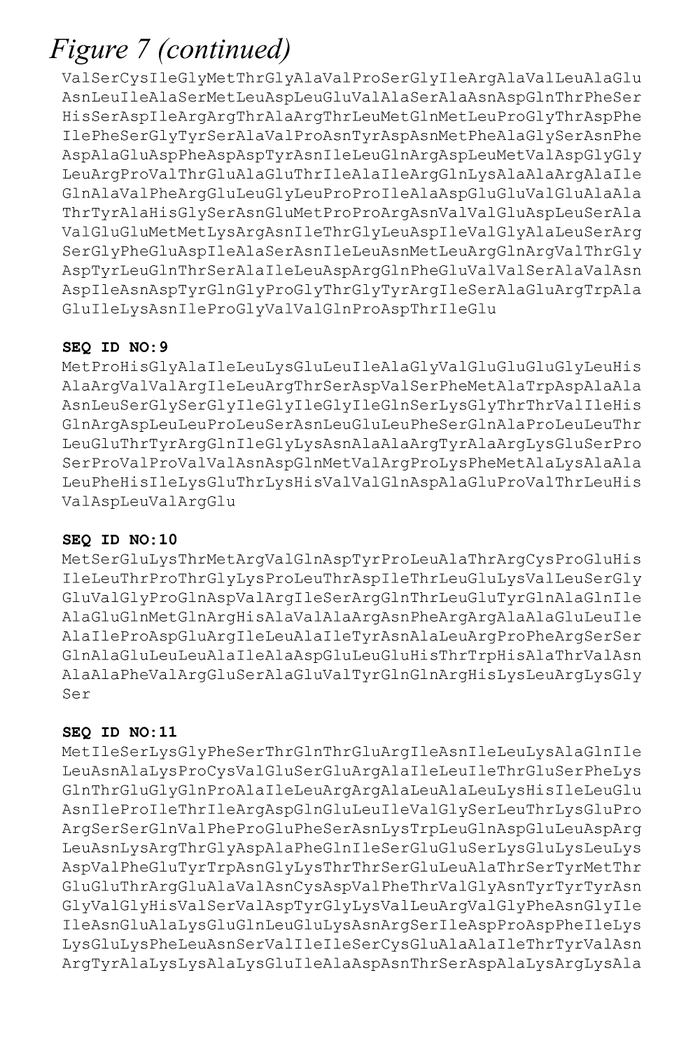

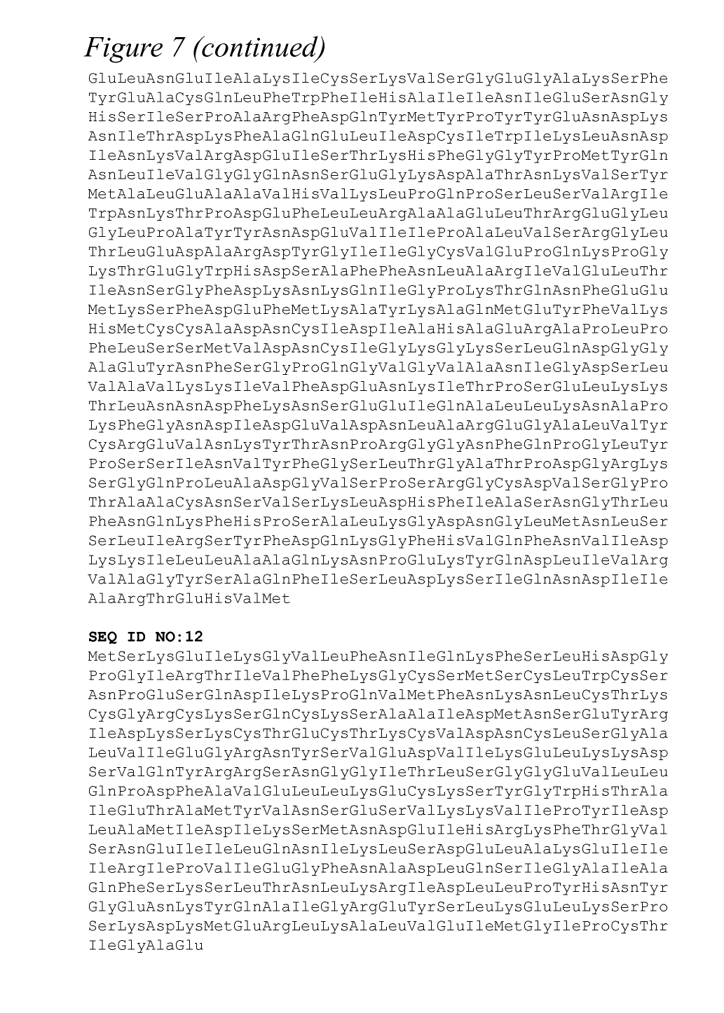

We reasoned that the decreased diol dehydratase activity is mainly due to plasmid incompatibility or pathway enzyme incompatibility. The expression of the diol dehydratase was disturbed by co-replication of pYY93 with pRJ11 (FIG. 3B), since expressing the diol dehydratase with only one plasmid (pYY93) was acceptable. We propose two different expression strategies to address this issue by integrating the 1-propanol pathway into only one plasmid. We will use the plasmid pYY93 (FIG. 3B) carrying the genes ppdABC as the starting construct. The 1,2-propanediol pathway that included the genes mgsA, ydjG, and budC will be further inserted downstream of the genes ppdABC. As shown in FIG. 5, the first expression approach contains only one promoter (pLlacO1) in front of the genes ppdABC, one terminator (T0) behind the gene budC and a RBS site located right before each of the structural genes. The 1-propanol pathway will be expressed as an entire operon. The second expression strategy introduces a promoter before and a terminator after each gene to express them individually and simultaneously. After constructing these two plasmids, we will transfer them into wild type E. coli BW25113 respectively for fermentation tests.

We expect this method to be successful, particularly given that we have demonstrated the diol dehydratase can completely convert 1,2-propanediol to 1-propanol. Similar tasks have been successfully accomplished in our lab. With one or both of the strategies we expect to decrease intermediate 1,2-propanediol accumulation and produce around 0.8 g/L 1-propanol in wild type E. coli BW25113. The following alternative approaches may also be employed: varying plasmid copy number by using different replicons to adjust enzyme expression; varying promoter strength by using different promoters to optimize gene transcription; and/or replacing the native genes with codon-optimized synthetic genes to improve enzyme translation in E. coli. Fourth, the diol dehydratase can be expressed as a fusion protein to balance the expression of each subunit and improve its activity in E. coli. Last, the diol dehydratase can be expressed in an E. coli strain independently and the generated strain used to convert the produced 1,2-propanediol via co-cultivation or sequential cultivation. It should be noted that these approaches can be combined to achieve the optimal solution.

### Example 3

### Manipulating Cell Metabolism for Efficient Fermentative Production of 1-Propanol

To achieve efficient production (high yield and high titer) of 1-propanol from glucose, we aim at manipulating E. coli natural metabolism to divert carbon flux towards 1-propanol formation, eliminating competing fermentative pathways to minimize both carbon and reducing power leakage, and providing an extra reducing source to boost 1-propanol titer. When glucose goes though glycolysis, it is split into equal mole amounts of DHAP and GAP. The triose phosphate isomerase encoded by the gene tpiA catalyzes the reversible conversion between these two compounds. Since glycolytic activity is dominant, the carbon flux is dragged from DHAP to glycolysis via GAP. Therefore, in order to retain the precursor, DHAP for 1-propanol generation, we will knock out the gene tpiA. We also reason that a tpiA knockout may not be sufficient, since glucose can also be metabolized through Entner-Doudoroff pathway only generating GAP (FIG. 6). This pathway is made up of four enzymes encoded by the genes zwf, pgl, edd, and eda. Therefore, we will further delete zwf to force glucose to only go through glycolysis and prevent any possible carbon leakage from Entner-Doudoroff pathway.

As shown in Example 1, lactate was observed to be the major by-product (over 7 g/L). To prevent lactate generation we will further disrupt ldhA from the E. coli genome. In addition, lactate can also be generated from methylglyoxal by an E. coli native detoxifying mechanism, glyoxalase system, which catalyzes two sequential reactions for the conversion of methylglyoxal to lactate (FIG. 6). The involved enzymes are encoded by gloA and gloB. Since deletion of gloA was reported to be sufficient to deactivate this mechanism, we will further knock out gloA to completely avoid the accumulation of lactate. To prevent carbon flux from going into other fermentative products such as ethanol and succinate, we will further disrupt the corresponding genes including adhE and frd. The gene knockouts will be carried out with either the lamda Red recombinase/FLP system or P1 transduction of the Keio collection (Baba et al., Mol. Syst. Biol. 2006, 2:2006 0008; Datsenko and Wanner, Proc. Natl. Acad. Sci. USA 2000, 97:6640-6645). The final E. coli strain (ΔtpiA, Δzwf, ΔldhA, ΔgloA, ΔadhE, Δfrd) will be subject to the introduction of the optimized 1-propanol pathway and fermentation tests.

The experimental approaches involved have been well-established and the effects of knockout of single or multiple genes we proposed have also been confirmed (Atsumi et al., Nature 2008, 451:86-89; Shen et al., Appl. Environ. Microbiol. 2011, 77:2905-2915). We expect to achieve a yield of more than 80% of theoretical maximum (one mole 1-propanol/mole glucose) with the engineered strain. The titer may be 1-2 g/L. To further improve the titer, we propose to introduce formate as an extra reducing source in the medium and express a formate dehydrogenase in the engineered strain to extract NADH from formate for 1-propanol generation. The gene fdh from Candida boidinii will be codon optimized for this purpose. With these efforts, we expect to improve the titer up to 5-10 g/L without hurting the yield. Note that formate can be potentially regarded as a sustainable reducing source since an electrochemical process of formate production from carbon dioxide has been developed (Li and Oloman, J. Appl. Electrochem. 2006, 36:1105-1115). The success of using formate as a reducing source for biofuel generation was also reported recently (Li et al., Science 2012, 335:1596). In case other metabolites are still produced as by-products, once any one is detected to be substantial (>0.5 g/L) we will look into the corresponding pathway to inactivate the related gene.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A microbe metabolically engineered to comprise at least one metabolic pathway for the production of 1-propanol from a 1,2-propanediol intermediate.

2. The microbe of claim 1 which produces 1-propanol using glucose as a carbon source.

3. The microbe of claim 1 metabolically engineered to overexpress an enzyme having methylglyoxal synthase activity.

4. The microbe of claim 1 metabolically engineered to overexpress an enzyme having secondary alcohol dehydrogenase activity.

5. The microbe of claim 5 wherein the secondary alcohol dehydrogenase comprises a diol dehydrogenase.

6. The microbe of claim 1 metabolically engineered to overexpress an enzyme having primary alcohol dehydrogenase activity.

7. The microbe of claim 6 wherein the enzyme having primary alcohol dehydrogenase activity comprises a methylglyoxal reductase.

8. The microbe of claim 6 wherein the enzyme having primary alcohol dehydrogenase comprises a lactaldehyde reductase.

9. The microbe of claim 6 wherein the primary alcohol dehydrogenase is native to the microbe.

10. The microbe of claim 1 comprising a first vector comprising a polynucleotide encoding at least one enzyme in a 1,2-propanediol pathway, the enzyme selected from one having methylglyoxal synthase activity, one having secondary alcohol dehydrogenase activity, and one having primary alcohol dehydrogenase activity.

11. The microbe of claim 10 wherein the first vector encodes methylglyoxal synthase, a methylglyoxal reductase, and a diol dehydrogenase.

12. The microbe of claim 10 wherein the first vector encodes an enzyme having methylglyoxal synthase activity and an enzyme having secondary alcohol dehydrogenase activity, wherein the enzyme having secondary alcohol dehydrogenase activity is a diol dehydrogenase or a glycerol dehydrogenase.

13. The microbe of claim 10 wherein the first vector encodes an enzyme having methylglyoxal synthase activity, an enzyme having secondary alcohol dehydrogenase activity, and an enzyme having primary alcohol dehydrogenase activity, wherein the enzyme having secondary alcohol dehydrogenase activity is a diol dehydrogenase or a glycerol dehydrogenase, and wherein the enzyme having primary alcohol dehydrogenase activity is a lactaldehyde reductase.

14. The microbe of claim 1 comprising two metabolic pathways for the production of the intermediate, 1,2-propanediol.

15. The microbe of claim 1 metabolically engineered to overexpress an enzyme having diol dehydratase activity.

16. The microbe of claim 15 wherein the enzyme having diol dehydratase activity is selected from a propanediol dehydratase and a glycerol dehydratase.

17. The microbe of claim 1 metabolically engineered to overexpress an enzyme having 1-propanal reductase activity.

18. The microbe of claim 1 which is a prokaryotic cell.

19. The microbe of claim 18 which is an E. coli cell.

20. The microbe of claim 19 wherein the E. coli cell comprises an enzyme having primary alcohol dehydrogenase activity, wherein the primary alcohol dehydrogenase is a lactaldehyde reductase, and wherein the lactaldehyde reductase is native to the prokaryotic cell.

21. The microbe of claim 20 wherein the E. coli cell comprises an enzyme having primary alcohol dehydrogenase activity, wherein the primary alcohol dehydrogenase is a lactaldehyde reductase, and wherein the lactaldehyde reductase is heterologous to the prokaryotic cell.

22. The microbe of claim 20 wherein the E. coli cell comprises an enzyme having primary alcohol dehydrogenase activity, wherein the primary alcohol dehydrogenase is a 1-propanal reductase, and wherein the 1-propanal reductase is native to the prokaryotic cell.

23. The microbe of claim 20 wherein the E. coli cell comprises an enzyme having primary alcohol dehydrogenase activity, wherein the primary alcohol dehydrogenase is a 1-propanal reductase, and wherein the 1-propanal reductase is heterologous to the prokaryotic cell.

24. A method for producing 1-propanol comprising culturing the microbe of claim 1 under conditions suitable to produce 1-propanol

25. The method of claim 24 further comprising isolating the 1-propanol.

26. The method of claim 24 comprising culturing the microbe in low phosphate media.

27. The method of claim 24 comprising culturing the microbe under anaerobic conditions.
